(19) United States
(12) Patent Application Publication
(10) Pub. No.: US 2003/0170665 A1
(43) Pub. Date: Sep. 11, 2003

(54) Haplotype map of the human genome and uses therefor

(75) Inventors: Mark J. Daly, Arlington, VA (US); David M. Altshuler, Brookline, MA (US); Eric S. Lander, Cambridge, MA (US); John D. Rioux, Cambridge, MA (US); Stacey B. Gabriel, Lexington, MA (US); Stephen Schaffner, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA

(21) Appl. No.: 10/213,272

(22) Filed: Aug. 5, 2002

Related U.S. Application Data
(60) Provisional application No. 60/310,055, filed on Aug. 4, 2001.
(60) Provisional application No. 60/381,189, filed on May 16, 2002.

(51) Int. Cl. C12Q 1/68; G06F 19/00; G01N 33/48; G01N 33/50

(52) U.S. Cl. 435/6; 702/20

(57) ABSTRACT

A method of producing a haplotype map which can be used to select an optimal set of single nucleotide polymorphic sites for examination in a subsequent genotyping study is disclosed, along with the map so produced.

### RELATED APPLICATION

This application claims the benefit of U.S. Provisional Application No. 60/310,055, filed on Aug. 4, 2001 and U.S. Provisional Application No. 60/381,189 filed on May 16, 2002. The entire teachings of both referenced provisional applications are incorporated herein by reference.

### BACKGROUND OF THE INVENTION

Defining the relationship between genetic make-up and a corresponding phenotype, e.g., predisposition to disease, has long been an important research topic. A clear appreciation of an individual's likelihood of developing a particular disease can provide both information and motivation for developing measures to prevent or delay onset of the disease, thus resulting in an improved quality of life. While studies utilizing family-based study approaches have been very successful for assessing predispositions to monogenic diseases, the complexity of common diseases renders them poor candidates for this type of approach (Goldstein, David B., 2001, Nature Genetics, 29(2):109-111).

Association studies which compare genotypic and phenotypic variation in populations to identify correlations indicating genetic risk factors are likely to prove more useful for defining the relationship in complex diseases. Many association studies examine allelic associations (also termed Linkage Disequilibrium (LD)). LD is defined as the non-random association of alleles at linked loci. Various methods of calculating LD have been employed, but most rely on measuring the difference between the observed frequency of the co-occurrence of two alleles with the expected frequency of their co-occurrence in a population. LD testing is typically carried out as a comparison of marker frequencies between individuals affected with a disease and unaffected control individuals. Although some success has been achieved using LD analysis for monogenic diseases, effectively utilizing the measure for complex disease states, particularly those with multiple loci, has proved difficult (Jorde, L. B., 2002, Genome Research 10(10):1435-1444).

Traditional LD analysis based on individual genetic markers often yields an erratic, non-monotonic picture in complex disease states because the power to detect association in such studies is affected by the specific properties of the individual marker. For example, past historical events such as admixture, genetic drift, multiple mutations and natural selection, can disturb the relationship between LD and inter-locus physical distance. Moreover, locus heterogeneity further complicates the analysis for complex diseases. Thus, although such analysis can indicate correlations between the markers and the disease, important localization information is often obscured by the properties of the markers.

Various methods of correcting for the bias introduced by marker properties have been employed, but none have been fully successful. In particular, much effort has been expended attempting to establish consistent patterns for LD in various populations. However, the noted patterns appear extremely variable, with patterns differing significantly between both genomic regions and populations, suggesting that a complete analysis of the genome could be required for effective applications of association methods relying on LD (Clark, A. G. et al., 1998, American Journal Human Genetics, 63:595-612; Paulussen, A. et al., 2000, Pharmacogenetics 10:415-424).

Therefore, methods of effectively conducting meaningful association studies for complex diseases would be extremely useful.

### SUMMARY OF THE INVENTION

The present invention is based, at least in part, on the recognition that the human genome is composed of discrete haplotype blocks of tens to hundreds of kilobases, each with strikingly limited diversity, bounded with sites of recombination with much greater diversity. The discrete haplotype blocks are segments of various sizes over which as little historical recombination is observed as is, for example, typical of very closely linked sites (those separated by less than 1,000 bp). Within each such block, haplotype diversity is typically extremely limited, with an average of three to six common haplotypes that together comprise, on average, 90% of all chromosomes in the population sample. The blocks are highly similar across population samples, with both their boundaries and specific haplotypes typically shared among the groups. A comprehensive catalogue of common haplotype blocks can provide a foundation to systematically test the role of common genetic variation in human disease.

The invention relates to a method of constructing a haplotype map comprising discrete haplotype blocks bounded by sites of recombination which can be used to readily select an optimal set of single nucleotide polymorphic sites (SNPs) for examination in subsequent genotyping studies. A set of SNPs, e.g., 6-8 common markers, can be used to uniquely distinguish the major haplotypes in each discrete haplotype block. All or a portion of these SNPs can then be used in methods to identify an association between a phenotype and a haplotype, to localize the position of a disease-susceptibility locus of a disease, and to diagnose susceptibility to a disease. Thus, the invention relates to a method of constructing,(i.e., building, making) a haplotype map of any region of the genome based on the objective structure of haplotype blocks.

Thus, in one aspect the invention is directed to a haplotype map of a region of interest of the human genome comprising one or more discrete haplotype blocks bounded by one or more sites of recombination. In one embodiment, the boundaries of the discrete haplotype blocks are determined by calculating the normalized linkage disequilibrium, D′, of pairs of polymorphic markers. In a specific embodiment, the 95% confidence intervals of the D′ of the pairs of polymorphic markers are utilized in determining the boundaries of the discrete haplotype blocks. The pairs of polymorphic markers can have a minor allele frequency of about 5% (0.20). The information utilized to prepare the map can be obtained from a multiethnic population sample, from a monoethnic population sample, or any combination thereof In one embodiment, the discrete haplotype blocks comprise a number of major haplotypes selected from the group consisting of 2, 3, 4, 5 and 6. In a particular embodiment, the region of interest comprises chromosome 5q31.

In another aspect, the invention is directed to a method of producing a haplotype map of a region of interest of the human genome comprising determining the pattern of historical recombination across the region of interest and determining one or more discrete haplotype blocks bounded by one or more sites of recombination, thereby producing a haplotype map of the region of interest. In one embodiment, the boundaries of the discrete haplotype blocks are determined by calculating the normalized linkage disequilibrium, D′, of pairs of polymorphic markers. In a specific embodiment, the 95% confidence intervals of the D′ of the pairs of polymorphic markers are utilized in determining the boundaries of the discrete haplotype blocks. The pairs of polymorphic markers can have a minor allele frequency of about 5% (0.20). The information utilized to prepare the map can be obtained from a multiethnic population sample, from a monoethnic population sample, or any combination thereof. In one embodiment the discrete haplotype blocks comprise a number of major haplotypes selected from the group consisting of 2, 3, 4, 5 and 6. In a particular embodiment, the region of interest comprises chromosome 5q31.

In another aspect, the invention is directed to a method of selecting a set of single nucleotide polymorphic sites, SNPs, for use in genotyping studies of a genomic region of interest comprising identifying at least one SNP which distinguishes each major haplotype in each discrete haplotype block in a haplotype map of the genomic region of interest of the human genome, wherein the haplotype map comprises one or more discrete haplotype blocks bounded by one or more sites of recombination; and selecting a sufficient number of the SNPs from each discrete haplotype block for use in a genotyping study; thereby selecting a set of SNPs for use in genotyping studies of the genomic region of interest. In one embodiment, the genomic region of interest is a chromosome. The information utilized to prepare the map can be obtained from a multiethnic population sample, from a monoethnic population sample, or any combination thereof. In a particular embodiment, the SNPs consist of those with a minor allele frequency greater than about 5% (0.20). In particular embodiments, the discrete haplotype blocks comprise a number of major haplotypes selected from the group consisting of 2, 3, 4, 5 and 6.

In another aspect, the invention is directed to methods utilizing one or more sets of SNPs identified according to the methods of the invention for an association between a phenotype and a haplotype. In a particular embodiment, the number of members in the set of SNPs consists of the sum of the number of major haplotypes in each discrete haplotype block minus the number of discrete haplotype blocks. In another embodiment, the SNPs forming the set of SNPs are selected from some or all of the discrete haplotype blocks.

In another aspect, the invention is directed to a method of selecting a set of SNPs for use in genotyping human chromosome 5q31 comprising identifying at least one SNP which distinguishes each major haplotype in each discrete haplotype block in a haplotype map of chromosome 5q31 consisting of one or more discrete haplotype blocks bounded by one or more sites of recombination; and selecting a sufficient number of the SNPs from each discrete haplotype blocks to use in a genotyping study, thereby selecting a set of SNPs for use in genotyping studies of chromosome 5q31. The particular set of SNPs identified using the methods of the invention are described herein. The information utilized to prepare the map can be obtained from a multiethnic population sample, from a monoethnic population sample, or any combination thereof. In a particular embodiment, the SNPs consist of those with a minor allele frequency greater than about 5% (0.20). In particular embodiments, the discrete haplotype blocks comprise a number of major haplotypes selected from the group consisting of 2, 3, 4, 5 and 6.

In another aspect, the invention is directed to a method of identifying an association between a phenotype and a haplotype comprising assessing one or more sets of SNPs selected according to the methods of the invention for an association between a phenotype and a haplotype in the human chromosome 5q31. In a particular embodiment, the number of members in the set of SNPs consists of the sum of the number of major haplotypes in each discrete haplotype block minus the number of discrete haplotype blocks. In another embodiment, the SNPs forming the set of SNPs are selected from some or all of the discrete haplotype blocks. In a particular embodiment, the genotyping study is directed to methods of detecting susceptibility to Crohn Disease (CD).

In yet another aspect, the invention is directed to a method of identifying an association between a phenotype and a haplotype comprising identifying a set of SNPs which uniquely distinguishes a haplotype by selecting the members of the set from a haplotype map consisting of one or more discrete haplotype blocks spanned by one or more sites of recombination; and assessing the set of SNPs to identify an association between a phenotype and a haplotype. In particular embodiments, the set of SNPs uniquely distinguishes a haplotype which is identical to the haplotype of a comparison individual in a percentage selected from the group consisting of 95%, 93%, 90%, 87%, 85%, 83%, 80%, 77%, 75%, 70%, 67%, 65%, 60%, 57%, 55%, and 50%. In particular embodiments, the number of the members of the set of SNPs is selected from the group consisting of 4, 5, 6, 7, 8, 9, 10, 11, 12, 13, 14, 15, 16, 17, 18, 19 and 20.

In another aspect, the invention is directed to a method of identifying the location of a gene associated with a phenotype comprising identifying a set of SNPs which uniquely distinguishes a haplotype by selecting the members of the set from a haplotype map of a chromosomal region associated with the phenotype consisting of one or more discrete haplotype blocks bounded by one or more sites of recombination; and assessing the set of SNPs to identify an association between a phenotype and a haplotype, wherein identification of the association between the haplotype and the phenotype is indicative of the location of the gene. In particular embodiments, the set of SNPs uniquely distinguishes a haplotype which is identical to the haplotype of a comparison individual in a percentage selected from the group consisting of 95%, 93%, 90%, 87%, 85%, 83%, 80%, 77%, 75%, 70%, 67%, 65%, 60%, 57%, 55%, and 50%. In particular embodiments, the number of the members of the set of SNPs is selected from the group consisting of 4, 5, 6, 7, 8, 9, 10, 11, 12, 13, 14, 15, 16, 17, 18, 19 and 20. In a particular embodiment, the phenotype is disease susceptibility.

In another aspect, the invention is directed to a method of diagnosis for susceptibility to a disease comprising identifying a set of SNPs which uniquely distinguishes a haplotype in a chromosomal region associated with the disease by selecting the members of the set from a haplotype map of the chromosomal region consisting of one or more discrete haplotype blocks bounded by one or more sites of recombination; and assessing the set of SNPs to identify an association between the haplotype and the disease, wherein identification of the association between the haplotype and the disease is indicative of susceptibility to the disease. In particular embodiments, the set of SNPs uniquely distinguishes a haplotype which is identical to the haplotype of a comparison individual in a percentage selected from the group consisting of 95%, 93%, 90%, 87%, 85%, 83%, 80%, 77%, 75%, 70%, 67%, 65%, 60%, 57%, 55%, and 50%. In particular embodiments, the number of the members of the set of SNPs is selected from the group consisting of 4, 5, 6, 7, 8, 9, 10, 11, 12, 13, 14, 15, 16, 17, 18, 19 and 20. In a particular embodiment, the disease is CD.

### BRIEF DESCRIPTION OF THE DRAWINGS

The file of this patent contains at least one drawing in color. Copies of this patent with color drawings will be provided by the Patent and Trademark office upon request and payment of the necessary fee.

FIGS. 1A-1F are a set of graphs. FIG. 1A is a graph of LD between marker 26 (position indicated with an asterisk) and every other marker in the data set using D′. FIG. 1B is a graph of the use of multiallelic D′ to plot LD between the haplotype group assignment at the location of marker 26 and that assignment at the location of every other marker in the data set. FIG. 1C and FIG. 1D are graphs which repeat the comparisons of FIGS. 1A and 1B, respectively, but with respect to marker 61 in the map. FIG. 1E is a graph of the single marker transmission ratio (T/U) for the overtransmitted allele at each SNP. FIG. 1F is a graph which plots the transmission ratio of haplotype class A across the entire region (with the region implicated in disease risk highlighted below—roughly positions 400 kb-650 kb).

FIGS. 2A-2D depict the blocklike haplotype diversity at 5q31. FIG. 2A displays the common haplotype patterns in each block of low diversity. Dashed lines indicate locations where >2% of all chromosomes are observed to transition from one common haplotype to a different one. FIG. 2B indicates the percentage of observed chromosomes that match one of the common patterns exactly. FIG. 2C reports the percentage of each of the common patterns among untransmitted chromosomes. FIG. 2D reports the rate of haplotype exchange between the blocks estimated by the Hidden Markov Model (HMM). (Several markers at each end were not included in the block analysis, because although they provided evidence that the blocks did not continue, they were not adequate to build a first or last block. In addition, 4 markers fell between blocks and suggest that the recombinational clustering may not take place at a specific bp position but rather in small regions.)

FIG. 3 depicts linkage and LD mapping. The curves in the top graph show the linkage evidence in the 18 cM surrounding marker D5S2497, from the initial genomewide scan, for the different disease subgroups: The linkage mapping identifies the 18 cM peak. The entire 18 cM region was examined with 1 SSLP/0.35 cM. ALL, all IBD families; CD, CD-only families; CD16, early onset CD families (Rioux, J. D. et al., Am. J. Hum. Genet. 66: 1863-1870 (2000)). The vertical tick marks indicated the position of the markers in the genomewide linkage study (LD mapping—stage 1) and the numbers in red refer to the marker numbers used in Table 1. The density of SSLP markers was then increased in regions of LD. The vertical tick marks on the thin horizontal line below the graph represent the position of all 56 markers used in the first stage of LD mapping in the 296 CD trios. These markers are numbered (shown in red where space provided) in map order and correspond to the numbers used in the tables. The region with significant LD is expanded below. LD mapping—stage 2 confirms LD. The multilocus analysis identifies a 435 kb haplotype. The thick grey horizontal line depicts the sequence contigs (the numbers below indicating the length in kb), and the gaps between the two sequence contigs is represented by a break in the horizontal line. Above the thick grey line are the names and positions (indicated by red diamonds) of the microsatellite markers used in the 1^{st} and 2^{nd} stage of LD mapping in this region. The known genes in this genomic region are shown below the thick grey line. The positions and length of the exons are indicated by the vertical green bars (drawn to scale so not all exons are distinguishable), and gene symbols are written above each gene. The thick blue line below the genes represents the region where SNP discovery was performed by resequencing DNA samples (of known genes) from eight individuals (seven CD patients and one CEPH DNA control). No candidate risk alleles were identified. The genomic region in patients for SNP discovery were resequenced. The blue line is continuous where the discovery was performed on every base over a 285 kb contiguous region (“core” region) and dashed where the discovery was noncontiguous regions (“proximal” and “distal” regions). The red tick marks beneath the blue line indicated the positions of the SNPs which have alleles unique to the risk haplotype where: A,IGR2055a_{—}1; B, IGR2060a_{—}1; C, IGR2063b_{—}1; D, IGR2069a_{—}2; E, IGR2078a_{—}1; F, IGR2096a_{—}1; G, IGR2198a_{—}1; H, IGR2230a_{—}1; I, IGR2277a_{—}1; J, IGR3081a_{—}1; K, IGR3096a_{—}1; L, IGR3236a_{—}1 (see text and Table 5). SNP discovery identified 651 common SNPs and the SNPs were genotyped in CD trios. The significant SNPs identified were SNPs unique to risk haplotype extending 250 kb.

FIGS. 4A-4B depict multilocus haplotype results. The curves represent the extent of association to the CD phenotype observed over the 1 cM region surrounding IBD5 using the data from the microsatellite markers described in Table 2. The multi-locus LD was measured using TDT (squares; values on left-hand Y-axis) or by Pexcess (triangles; values on right-hand Y-axis). The tick marks along the X-axis represent the positions of each marker. The thick black line and marker names and numbers are as described in FIG. 3. The arrow points to the peak LD in this region, observed for the haplotype formed by the IRF1p1, Cah15a, and Cah17a markers (alleles 156, 373, and 140, respectively. FIG. 4A shows a two-locus haplotypes: results are shown for all pairs of adjacent markers where the data points are drawn at the midpoint between the two markers. FIG. 4B shows a three-locus haplotypes: results are shown for all possible combinations of adjacent markers where the data points are drawn at the position of the middle marker.

FIG. 5 depicts multi-point T/U plot for the IBD5 risk haplotype. This curve represents the transmitted to untransmitted ration (T/U) of the IBD5 risk haplotype identified with the high density SNP genotype information for the individuals in set C. Ancestral haplotype blocks were discovered in this region (kilobase positions are as per our 983 kb reference sequence) and multipoint TDT was performed.

FIGS. 6A and 6B are a set of graphs. FIG. 6A depicts the normalized allele frequency of candidate SNPs. The distribution is normalized to a constant number of chromosomes (n=64) randomly sampled) from the European, African-American, Asian, and Yoruban samples. Of candidate SNPs assayed in all four populations, both predicted alleles were observed in 89% of cases. FIG. 6B depicts an assessment of pairwise linkage disequilibrium across populations. The proportion of informative SNP pairs that display strong evidence for recombination is plotted at various intermarker distances. Between 9,860 and 13,980 SNP pairs were examined in each sample.

FIGS. 7A-7C are a set of graphs. FIGS. 7A-7B depict the scaffold analysis of Yoruban and African-American (A), and European and Asian (B) samples. The x-axis indicates the fraction of independent, informative marker pairs (within each region) displaying the strong evidence for recombination. The x-axis indicates the distance between the outermost marker pair defining the region. The three lines represent the distribution of LD for all pairs (without any filtering for the LD of flanking markers), and for regions meeting the empirically derived two and three marker criteria. FIG. 7C shows the relation of linkage disequilibrium to physical distance within haplotype blocks, as assessed by the mean value of the correlation coefficient (r²) and the mean value of D′. The marker pairs reported were not used to define the region as a block and, thus, represent an unbiased estimation of the relation between LD and distance within a block.

FIGS. 8A-8D are graphs which illustrate block characteristics across populations. FIG. 8A depicts the size (in kb) of all haplotype blocks found in the analysis. FIG. 8B depicts the proportion of all genome sequence spanned by blocks, binned according to the size of each block. FIGS. 8C and 8D summarize the haplotype diversity across all blocks. FIG. 8C shows the number of common (≧5%) haplotypes per block. FIG. 8D shows the fraction of all chromosomes representing a perfect match to one of these common haplotypes plotted as a function of the number of markers typed in each block.

FIGS. 9A-9E shows a comparison of blocks across population samples. FIGS. 9A-9D show the concordance of block assignments for adjacent SNP pairs, compared across populations. White bars show the fraction of concordant SNP pairs; black bars the proportion of discordant SNP pairs. Population samples are abbreviated as follows: EU, European sample; AS, Asian sample; AA, African-American sample; YR, Yoruban sample. FIG. 9E shows the distribution of haplotypes across populations.

FIGS. 10A-10E show the allele frequency scatter for pairs of populations. The corresponding Fₛₜ value is indicated on each plot. FIG. 10A shows the Yoruban population compared to the European population. FIG. 10B shows the European population as compared to the Asian population. FIG. 10C shows the Yoruban population compared to the Asian population. FIG. 10D shows the Yoruban population compared to the African-American population. FIG. 10E shows the composite European-Yoruban population compared to the composite Asian-Yoruban population.

FIGS. 11A-11D depict the block-like structure of linkage disequilibrium across four populations. Pairwise D′ values for pairs of markers within each population sample are represented. FIG. 11A depicts the Yoruban sample, FIG. 11B the African-American sample, FIG. 11C the European sample, and FIG. 11D the Asian sample. Block diagrams include SNPs with frequency >20% in the given population. Black squares indicate strong LD; white squares, strong evidence for recombination; gray squares all other uninformative comparisons examined in each sample.

FIG. 12 is a graph which depicts haplotype frequencies within blocks as estimated by the EM algorithm. Haplotype frequencies based on phased-data versus unphased data from the same individuals.

FIG. 13 shows the physical location and SNP coverage in 54 autosomal clusters.

### DETAILED DESCRIPTION OF THE INVENTION

Variation in the human genome sequence plays a powerful but poorly understood role in the etiology of common medical conditions. Because the vast majority of heterozygosity in the human population is attributable to common variants, and because the evolutionary history of common human diseases (which determined the allele spectrum for causal alleles) is not yet known, one promising approach is to comprehensively test common genetic variation for association with medical conditions (Lander, E. S. Science 274:536 (1996); Collins, F. S., et al., Science 278:1580 (1997); Risch, N., Science 273:1516 (1996). This approach is increasingly practical because 4 million (Sachidanandam et al., Nature 409:928 (2001); Venter, J. C., et al., Science 291: 1304 (2001) of the estimated 10 million (Kruglyak, L., Nature Genet 27:234 (2001)) common single nucleotide polymorphisms (SNPs) are already known.

The term “polymorphism” as used herein refers to the occurrence of two or more genetically determined alternative sequences or alleles in a population. Several different types of polymorphism have been reported. A restriction fragment length polymorphism (RFLP) as used herein means a variation in DNA sequence that alters the length of a restriction fragment, as described in Botstein et al., Am. J. Hum. Genet. 32:314-331 (1980). The restriction fragment length polymorphism may create or delete a restriction site, thus changing the length of the restriction fragment. RFLPs have been widely used in human and animal genetic analyses (see WO 90/13668; W090/11369; Donis-Keller, Cell 51:319-337 (1987); Lander et al., Genetics 121:85-99 (1989)). When a heritable trait can be linked to a particular RFLP, the presence of the RFLP in an individual can be used to predict the likelihood that the animal will also exhibit the trait.

Other polymorphisms take the form of short tandem repeats (STRs) that include tandem di-, tri- and tetra-nucleotide repeated motifs. These tandem repeats are also referred to as variable number tandem repeat (VNTR) polymorphisms. VNTRs have been used in identity and paternity analysis (U.S. Pat. No. 5,075,217; Armour et al., FEBS Lett. 307:113-115 (1992); Horn et al., WO 91/14003; Jeffreys, EP 370,719), and in a large number of genetic mapping studies.

The phrase “single nucleotide polymorphism” (SNP) as used herein refers to a polymorphism which takes the form of a single nucleotide variation between individuals of the same species. Such polymorphisms are the most common type of polymorphism. Some SNPs occur in protein-coding sequences, in which case one of the polymorphic forms may give rise to the expression of a defective or other variant protein and, potentially, a genetic disease. Other SNPs occur in noncoding regions. Some of these polymorphisms may also result in defective protein expression, e.g., as a result of defective splicing. Other SNPs have no phenotypic effects. The “SNP site” as used herein refers to the locus at which divergence occurs.

Testing common variants for association with disease phenotypes offers a promising approach to understanding the population basis of common medical conditions (Lander, E. S., Science 274:536-9 (1996); Risch, N., et al., Science 273:1516-7 (1996); Collins, F. S., et al., Science 278:1580-1 (1997)). Two strategies have been described for performing genetic association studies of human disease (Lander, E. S., et al., Science 265:2037-48 (1994)). In “direct” association studies, each putative causal variant is discovered and tested for association to disease in the population; this offers the greatest power, but requires the identification of each causal variant prior to association testing. “Indirect” association studies instead exploit the correlation between each disease-causing mutation and the chromosomal haplotype—the particular set of alleles carried on a single physical chromosome—on which it arose. Barring significant recombination or mutation since the shared ancestors of the current population, it should be possible to identify each ancestral haplotype and to test it as a unit for association to phenotype. Furthermore, with knowledge of these haplotypes, furthermore, it would be possible to select a subset of SNPs (Johnson, G. C., et al., Nat Genet 29:233-237 2001); Patil, N., et al., Science 294:1719-23 (2001)) that mark each ancestral segment, allowing it to be studied in the population in an efficient and powerful design.

Thus, for the most powerful design and interpretation of association studies of genotype and phenotype, it is necessary to understand the structure of haplotypes in the human genome. Haplotypes are the particular combinations of alleles observed in a population. When a new mutation arises, it does so on a specific chromosomal haplotype. The association between each mutant allele and its ancestral haplotype is disrupted only by mutation and recombination in subsequent generations. Thus, it should be possible to track each variant allele in the population by identifying (through the use of anonymous genetic markers) the particular ancestral segment on which it arose. Haplotype methods have contributed to the identification of genes for Mendelian diseases (Puffenberger, E. G., et al, Cell 79:1257 (1994); Kerem, B., et al, Science 245:1073 (1989); Hastbacka, J. et al., Nature Genet. 2:204 (1992)) and, recently, disorders that are both common and complex in inheritance (Rioux, J. D., et al., Nature Genet 29:223 (2001); Hugot, J. P. et al., Nature 411:603 (2001); Ogura, Y. et al, Nature 411:603 (2001). However, until now, the general properties of haplotypes in the human genome have remained unclear.

Phenotypic traits which can be indicative of a particular haplotype include symptoms of, or susceptibility to, diseases of which one or more components is or may be genetic, such as autoimmune diseases, inflammation, cancer, diseases of the nervous system, and infection by pathogenic microorganisms. Some examples of autoimmune diseases include rheumatoid arthritis, multiple sclerosis, diabetes (insulin-dependent and non-independent), systemic lupus erythematosus and Graves disease. Some examples of cancers include cancers of the bladder, brain, breast, colon, esophagus, kidney, leukemia, liver, lung, oral cavity, ovary, pancreas, prostate, skin, stomach and uterus. Phenotypic traits also include characteristics such as longevity, appearance (e.g., baldness, color, obesity), strength, speed, endurance, fertility, and susceptibility or receptivity to particular drugs or therapeutic treatments. Many human disease phenotypes can be simulated in animal models. Examples of such models include inflammation (see e.g., Ma, Circulation 88:649-658 (1993); multiple sclerosis (Yednock et al., Nature 356:63-66 (1992); Alzheimer's disease (Games, Nature 373:523 (1995); Hsiao et al., Science 250:1587-1590 (1990)); cancer (see Donehower, Nature 356:215 (1992); Clark, Nature 359:328 (1992); Jacks, Nature 359:295 (1992); and Lee, Nature 359:288 (1992); cystic fibrosis (Snouwaert, Science 257:1083 (1992)); Gaucher's Disease (Tybulewicz, Nature 357:407 (1992)); hypercholesterolemia (Piedrahita, PNAS 89:4471 (1992)); neurofibromatosis (Brannan, Genes & Dev. 7:1019 (1994)); Thalaemia & Shehee, PNAS 90:3177 (1993)); Wilm's Tumor (Kreidberg, Cell 74:679 (1993)); DiGeorge's Syndrome (Chisaka, Nature 350:473 (1994)); infantile pyloric stenosis (Huang, Cell 75:1273 (1993)); inflammatory bowel disease (Mombaerts, Cell 75:275 (1993)).

Many studies have examined allelic associations (most commonly referred to as “linkage disequilibrium” or LD) across one or a few gene regions. These studies have generally concluded that LD is extremely variable within and among loci and populations (Pritchard, J. P. et al, Am J Hum Genet 69:1-14 (2001); L. B. Jorde, Genome Res 10, 1435-1444. (2000); M. Boehnke, Nat Genet 25, 246-247 (2000). Thus, LD analysis based on individual genetic markers often yields an erratic, non-monotonic picture, because the power to detect association in such studies depends on specific properties of each marker such as its frequency and population history. However, when studies utilizing a higher density of markers over contiguous regions are examined (Daly, M. J., et al, Nat Genet 29:229-232 (2001); Jeffreys, A. J., et al, Nat Genet 29:217-222 (2001); Patil, N., et al, Science 294:1719 (2001)), a pattern of blocks of variable length over which only a few common haplotypes are observed punctuated by sites at which recombination could be inferred in the history of sample can be noted. For example, in one segment of the major histocompatibility complex (MHC) on chromosome 6, it has been directly demonstrated that “hotspots” (sites of recombination) of meiotic recombination coincided with boundaries between such blocks ( Jeffreys, A. J., et al, Nat Genet 29:217-222 (2001). However, prior to the present invention, it remained unclear whether a model haplotype structure could be extrapolated across the genome. The extent to which population samples could affect the model, the number of SNPs required to detect haplotype patterns in any one region and the model's ability to capture common sequence variation were also unknown. Additionally, other specific aspects of the model, for example, a method to determine the boundaries of the discrete haplotype blocks, were unknown.

The term “linkage” as used herein describes the tendency of genes, alleles, loci or genetic markers to be inherited together as a result of their location on the same chromosome. Linkage can be measured in various ways. “Linkage disequilibrium”, or LD”, as used herein, refers to the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium.

A marker in linkage disequilibrium can be particularly useful in detecting susceptibility to disease (or other phenotype) notwithstanding that the marker does not cause the disease. For example, a marker X that is not itself a causative element of a disease, but which is in linkage disequilibrium with a gene Y that is a causative element of a phenotype, can be used to indicate susceptibility to the disease in circumstances in which the gene Y may not have been identified or may not be readily detectable.

Linkage can be analyzed by calculation of LOD (log of the odds) values. A lod value is the relative likelihood of obtaining observed segregation data for a marker and a genetic locus when the two are located at a recombination fraction (θ), versus the situation in which the two are not linked, and thus segregating independently (Thompson & Thompson, Genetics in Medicine (5th ed, W. B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (θ), ranging from θ=0.0 (coincident loci) to θ=0.50 (unlinked). Thus, the likelihood at a given value of θ is: probability of data if loci linked at θ to probability of data if loci unlinked. The computed likelihoods are usually expressed as the log10 of this ratio (i.e., a lod score). For example, a lod score of 3 indicates 1000:1 odds against an apparent observed linkage being a coincidence. The use of logarithms allows data collected from different families to be combined by simple addition. Computer programs are available for the calculation of lod scores for differing values of θ (e.g., LIPED, MLINK (Lathrop, Proc. Nat. Acad. Sci. (USA) 81:3443-3446 (1984)). For any particular lod score, a recombination fraction may be determined from mathematical tables. See Smith et al., Mathematical tables for research workers in human genetics (Churchill, London, 1961); Smith, Ann. Hum. Genet. 32:127-150 (1968). The value of θ at which the lod score is the highest is considered to be the best estimate of the recombination fraction.

Positive lod score values suggest that the two loci are linked, whereas negative values suggest that linkage is less likely (at that value of θ) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

As described herein, the haplotype structure of 54 autosomal regions each with an average size of 250,000 base pairs (bp) distributed across the human genome (covering 13.4 Mb or 0.4% of the genome) has been systematically characterized by genotyping a high density of markers in a large and diverse sample: 400 chromosomes drawn from 275 individuals in four population groups: European, Asian, African and African-American. Regions were selected to fit two criteria: that they be evenly spaced throughout the genome and that they contain an average density (in a core region of 150 kilobases (kb) of one candidate SNP discovered by The SNP Consortium (TSC) every two kb. It is demonstrated, herein, that discrete haplotype blocks are a general feature of the human genome, and that they can be objectively defined based upon the underlying structure of historical recombination across each region. The data indicate that the majority of the human genome (more that 75% of the genome is estimated to exist in blocks larger than 10 kb in all populations) can be objectively parsed into these haplotype blocks—segments of various sizes over which as little historical recombination is observed as is, for example, typical of very closely linked sites (those separated by less than 1,000 bp). Within each block, there is limited haplotype diversity, with an average of three to six common (≧5%) haplotypes that capture ≈90% of all chromosomes in the population. The blocks are highly similar across population samples, with both their boundaries and specific haplotypes often shared among the groups. The sites of historical recombination and specific haplotypes observed in the European and Asian samples are largely a subset of those seen in the Yoruban and African-American samples, with the most frequent recombinant haplotypes present in the African samples being most likely to be pan-ethnic. These haplotype blocks are estimated to have a mean size of 22 kb in a European and Asian sample and 11 kb in an African (Yoruban) and African-American sample.

Thus, a comprehensive catalogue of common haplotype blocks is useful and can provide a foundation to systematically determine the role of common genetic variation in human disease. This would make it possible to scan the genome for the presence of associated disease mutations without having to discover and test each SNP individually. Once a disease-associated haplotype is found, it can be intensively studied to identify the causal mutations it carries.

The phrases “discrete haplotype block” and “haplotype block” are used interchangably herein. The phrases, as used herein, refer to a region over which historical recombination is identical or similar to that typically observed for marker pairs separated by very short distances, for example, <500 bp, <1,000 bp, <1500 bp and does not substantially decline as a function of the distance separating marker pairs. The history of recombination between a pair of markers, e.g., RFLPs, STRs, VNTRs and SNPs, can be measured using any method known in the art including the various methods of measuring allelic association or LD. The LD between two markers, can, for example, be estimated with the use of the normalized measure of allelic association D′ (Daly, M. J., et al, Nat Genet 29:229-232 (2001); Lewontin, R. C., Genetics 49:49 (1964). Most typically, D′ is calculated using the normalized linkage disequilibrium measurement D′ of Lewontin. (R. C. Lewontin, Genetics 49:49-67 (1964)). This measurement can be represented by the formula: D′_{lJ}=D_{lJ}/Dₘₐₓ where _Dₘₐₓ=min└pₗq_{J}, (1−pₗ)(1−q_{J})┘ when _D_{lJ}<0. D′ values are known to fluctuate upward when a small number of samples or rare alleles are examined. This fluctuation can be remedied by, for example, relying on the confidence bounds on D′ rather than point estimates. Confidence bounds, both upper and lower, can be in any range including >75%, >80%, >85%, >90%, >92%, >93%, >94%, >95%, >96%, >97%, >98% or >99%. Typically, such ranges are >95%.

Pairs of markers are said to be in “strong LD” herein if the one-sided upper 95% confidence bound on D′ is >98% (a level consistent with a lack of historical recombination) and the lower bound is above 0.70. Conversely, pairs of markers are said to exhibit “strong evidence for historical recombination” herein if the upper confidence bound on D′ is less than 0.9. “Informative markers” are those markers with a minor allele frequency of at least 5% (0.20) which either exhibit strong LD or strong evidence for historical recombination.

These results point towards the feasibility and desirability of building a haplotype map of the human genome. Within haplotype blocks, common sequence variation can be captured by a small number of SNPs (Johnson, G. C., et al., Nat Genet 29:233-237 (2001)) that efficiently represent the haplotype variation across a large region. For a block containing N haplotypes, at most (N−1) SNPs will be required to differentiate all common haplotypes. Of course, a comprehensive description of the blocks and common haplotypes in the human population can require a high density of polymorphic markers. Across much of the genome and where blocks are large, the public SNP map is already adequate to map out blocks and define haplotypes. Where blocks happen to be small and the current SNP map does not yet provide adequate marker density, additional SNPs will be required; but given the thousand-fold increase in known SNPs over the last three years (Sachidanandam, R., et al., Nature 409:928-933 (2001); Wang, D. G., et al., Science 280:1077-1082 (1998)), achieving an adequate marker density across the entire human genome can be readily achieved. Identification of tnew SNPs can be readily accomplished using methods known in the art. Detailed knowledge of human haplotype structure can provide rich information about human history and genome evolution, and provide a foundation for population-based association studies to assess the bulk of human genome sequence variation for a contribution to disease.

Information can be obtained from any sample population to produce a map of the invention. “Information” as used herein in reference to sample populations is intended to encompass data regarding frequency and location of polymorphisms and other data such as background and health information useful in genotype studies and the methods and maps of the invention described herein. In some cases it can be desirable to utilize a multiethnic population sample. Such a sample can include a total random sample in which no data regarding ethnic origin is known. Alternatively, such a sample can include samples from two or more groups with differing ethnic origins. Such multiethnic samples can also include samples from three, four, five, six or more groups. Ethnic origins can be, for example, European, Asian, African or any other ethnic classification or any subset or combination thereof. The population samples can be of any size including 5, 10, 15, 20, 25, 30, 35, 40, 50, 75, 100, 125, 150 or more individuals.

In other cases it can be desirable to utilize a monoethnic sample in which all members of the population have the same ethnic origin. Ethnic origins can be, for example, European, Asian, African or any other ethnic classification or any single subset or combination thereof. The population samples can be of any size including 5, 10, 15, 20, 25, 30, 35, 40, 50, 75, 100, 125, 150 or more individuals.

Information for producing a map of the invention can also be obtained from multiple sample populations. Such information can be used concurrently or sequentially. For example, studies can be performed using monoethnic population samples. The results of these studies can then be utilized with the results of a multiethnic study. Alternatively, the results from the monoethnic study can be combined to form a multiethnic study.

As described herein, a 500 kb region on human chromosome 5q31 is implicated as containing a genetic risk factor for Crohn disease (CD). High-density SNP discovery across the region was performed and 97 common SNPs in 129 trios from a European-derived population from Toronto, Canada were genotyped. The study focused primarily on those SNPs with minor allele frequency >5% (0.20). The results describe 258 chromosomes transmitted to CD patients and 258 normal, untransmitted chromosomes. These results also show a picture of discrete haplotype blocks (of tens to hundreds of kb), each with strikingly limited diversity, punctuated by apparent sites of recombination.

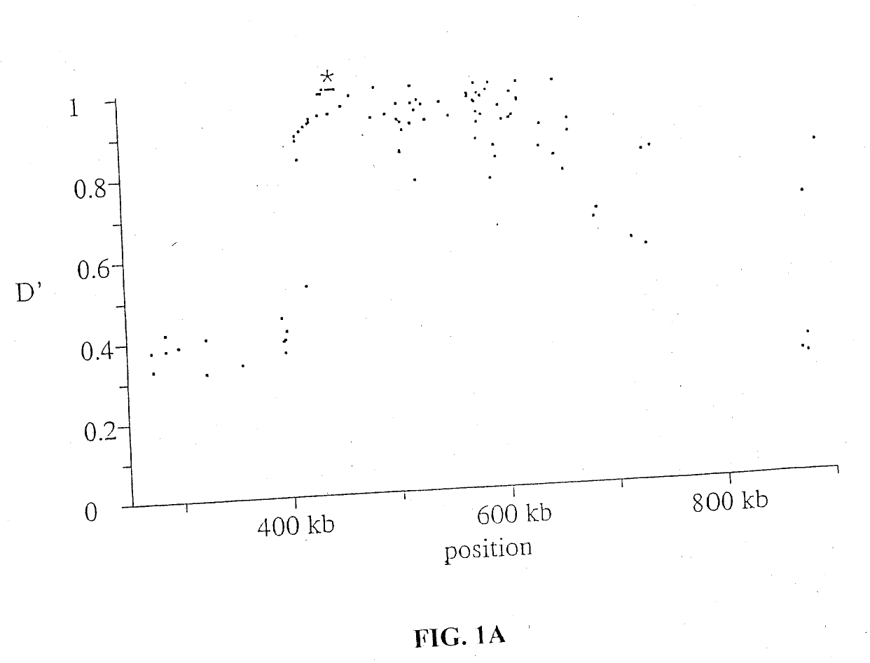

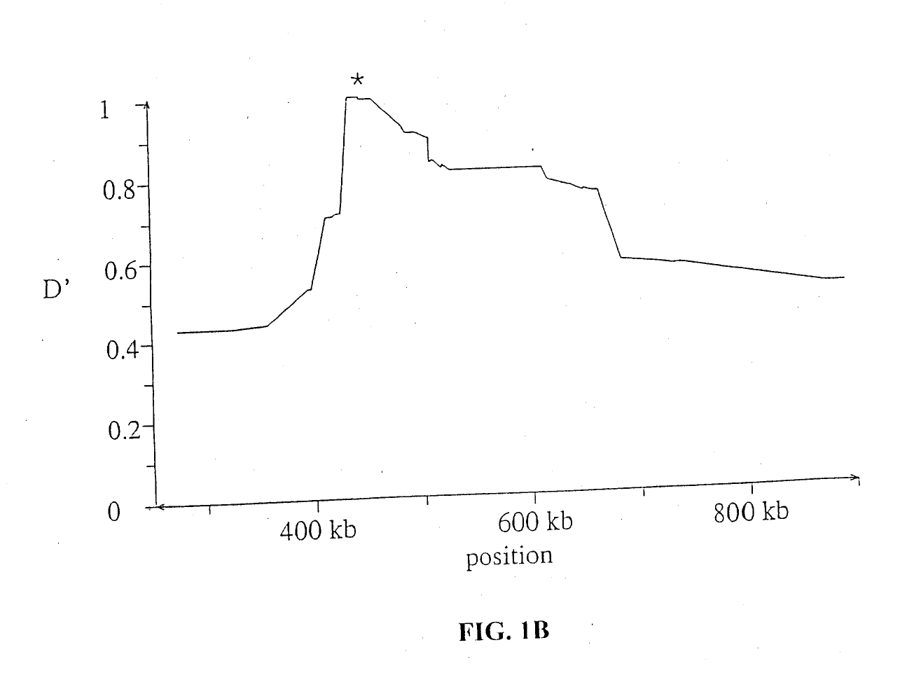

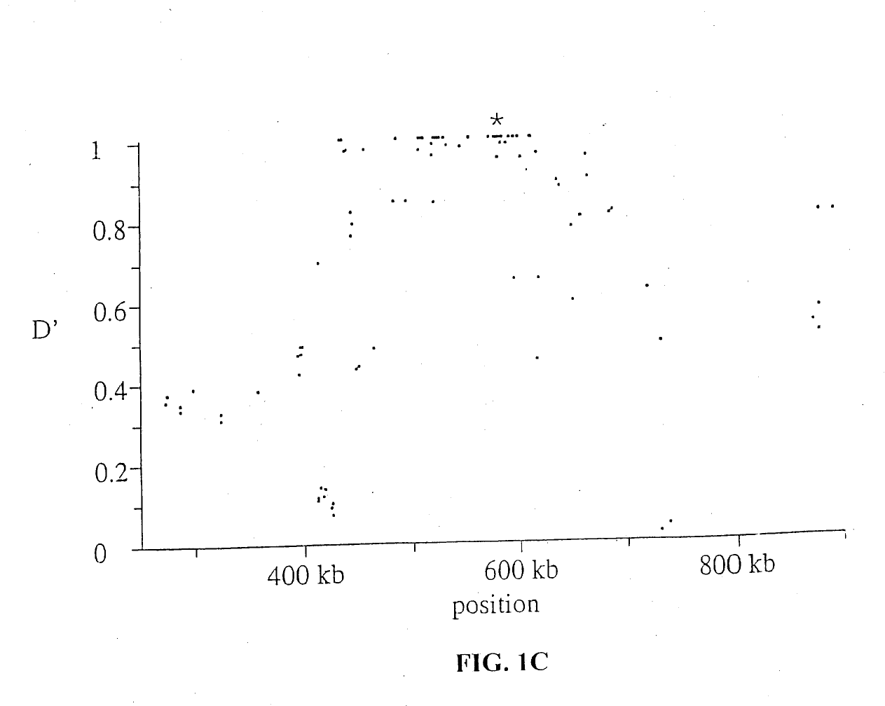

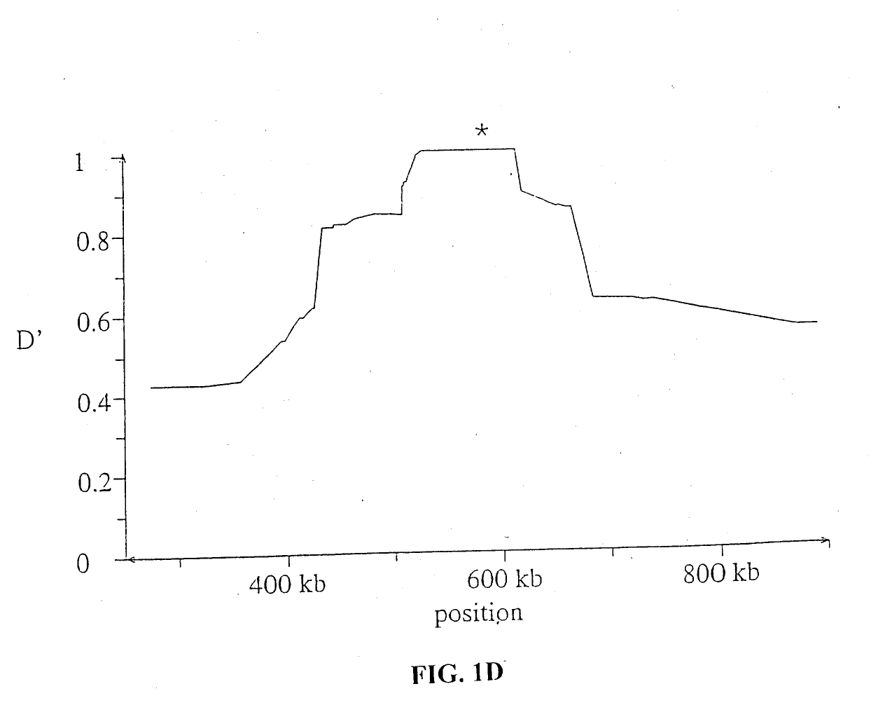

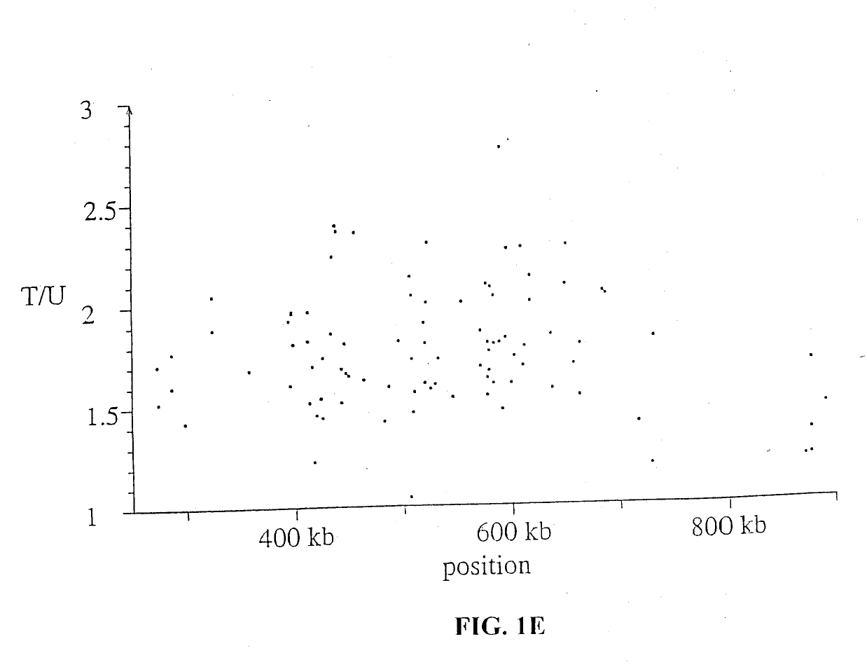

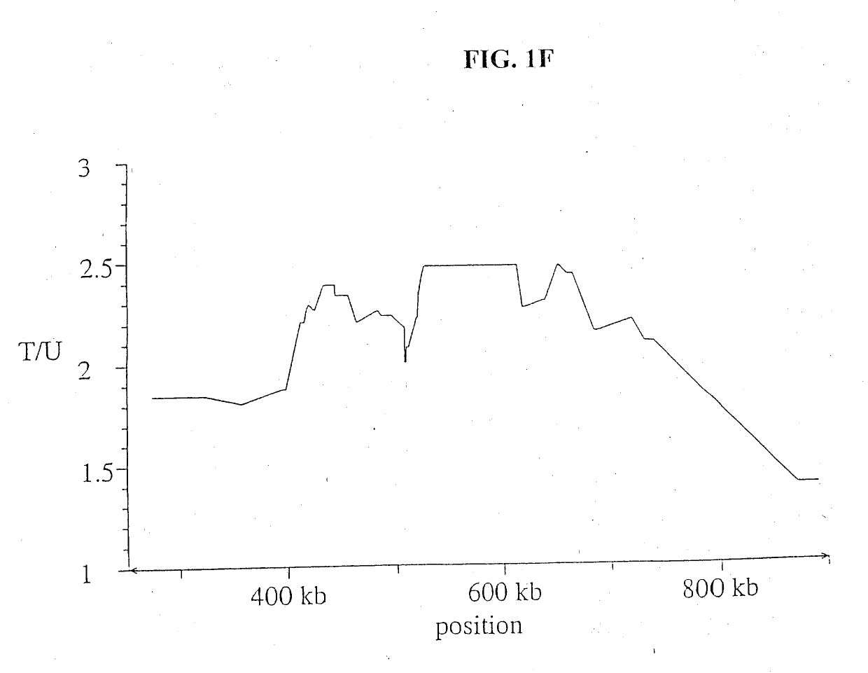

The genotype data provide a high resolution picture of the pattern of genetic variation across a large genomic region, with a marker density of 1 SNP every ˜5 kb. The traditional approach to analyzing such data has been to perform single-marker analysis, both to study disease association (marker vs. disease) and linkage disequilibrium (marker vs. marker). Examples of such analysis are shown in FIGS. 1A-1E. Although there are clearly many strong correlations, the picture is noisy and unsatisfying: important localization information is obscured by properties of the markers not relevant to these issues.

This association (as with allele sharing between affected siblings at loci with weak effect) is not expected to display the monotonicity or peak specificity that the simple plots of LD will. However the contrast between the results of the single marker analysis (in which the vast majority of markers show little if any evidence of association) and the haplotype based analysis (where haplotype across an entire 250 kb region is identified as being overtransmitted at >2-1) is still striking.

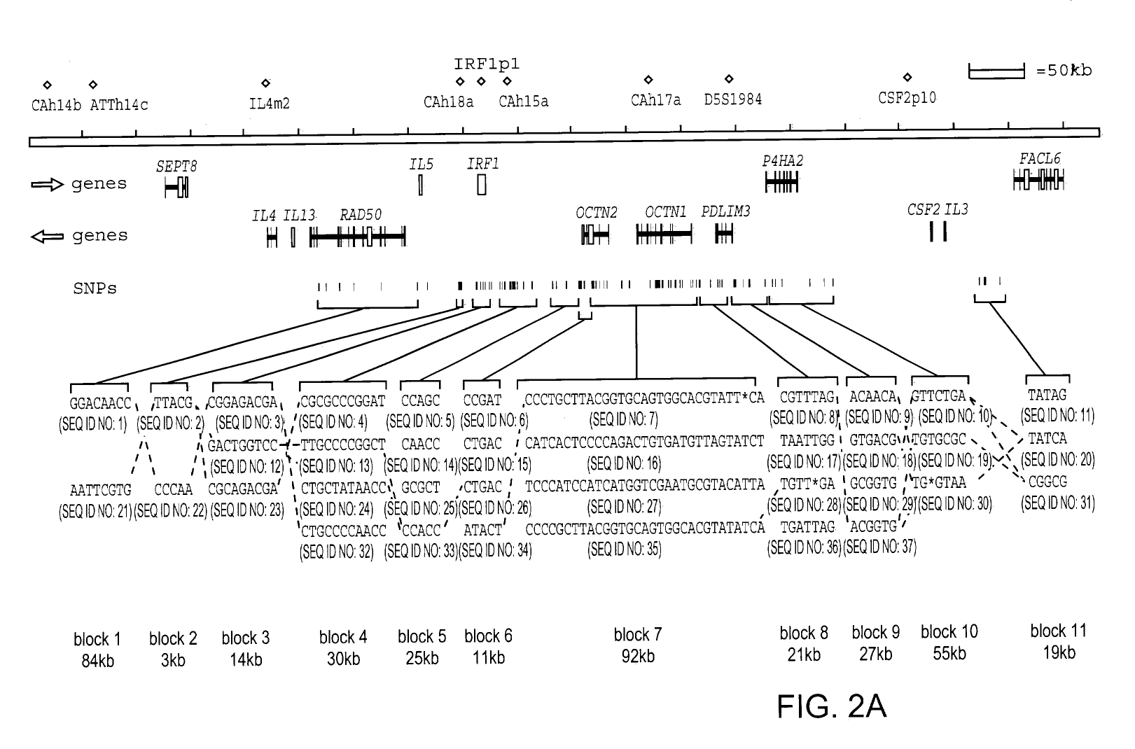

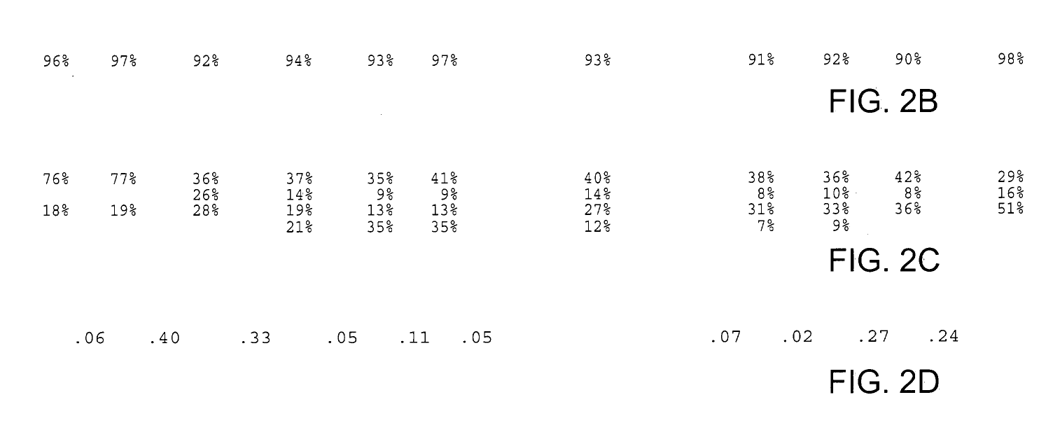

To obtain a clearer picture, the study of chromosome 5q31 focused on systematically identifying the underlying haplotypes. It became evident that the region could be largely decomposed into discrete haplotype blocks, each with striking lack of diversity (FIG. 2). Although the initial focus was on untransmitted control chromosomes; however, the same haplotype structure was seen in the chromosomes transmitted to CD patients, with the only difference being that one of the haplotypes was enriched in frequency reflecting its association to CD. Because the underlying ancestral structure is the same in both groups, combined data from all chromosomes (transmitted and untransmitted) is presented here. All of the underlying data for both groups are available at the website noted in the methods section which follows.

The haplotype blocks in this region span 10 to 100 kb and contain multiple (5 or more) common SNPs. The blocks have only a handful of haplotypes (2-4), which show no evidence of being derived from one another by recombination and which together account for nearly all chromosomes (>90% in all cases) in the sample. For example, an 84 kb block shows only two distinct haplotypes that together account for 97% of the observed chromosomes (Table 1). The lack of diversity is readily seen from the fact that the probability that a haplotype block is homozygous (for the SNPs genotyped) ranges from 30-70%.

The discrete blocks are separated by intervals in which multiple independent historical recombination events appear to have occurred, giving rise to greater haplotype diversity for regions spanning the blocks. Such recombination events are denoted in FIG. 2 by lines connecting haplotypes. The recombination events appear to be clustered, with multiple obligate exchanges needing to have occurred between blocks but little or no exchange within blocks. For example, in the aforementioned 84 kb block (Table 1), not a single apparent recombinant between the two major haplotypes was observed (despite the fact that such a recombinant would be readily evident because the haplotypes differ at all SNPs examined). The clustering is suggestive of local hotspots of recombination (Templeton, A. R. et. al., Am. J. Hum. Genet. 66:69-83 (2000); Jeffreys, A. J., et al., Hum. Mol. Genet. 9:725-733 (2000); Smith, R. A. et al., Blood 92:4415-4421 (1998). Although there is detectable recombination between blocks, it is modest enough that there is clear long-range correlation (that is, linkage disequilibrium) among blocks. The haplotypes at the various blocks can be readily assigned to one of four ancestral long-range haplotypes (A, B, C, D). Indeed, 38% of the chromosomes studied carried one of these four haplotypes across the entire length of the region.

A mathematical approach to formally define the block structure was developed by using a Hidden Markov Model (HMM). The HMM simultaneously assigns every position along each chromosome to an ancestral haplotype (in this case, A, B, C, or D) and estimates the maximum-likelihood values of the “historical recombination frequency” (⊖) between each pair of markers. The quantity ⊖ provides a convenient summary of the degree of haplotype exchange across inter-marker interval and relates directly to the conventional measures of LD such as D′ (methods). An alternative measure is the joint probability of homozygosity (Sved, J. A., Theor. Pop. Biol. 2:125-141 (1971)). In the case at hand, the discrete block structure is evident from the fact that e is estimated at <1% for 73 of the inter-marker intervals, 1-4% for the 14 of the intervals, and >4% for only 9 of the intervals.

Focusing on haplotype blocks in this chromosome also greatly clarifies LD and association analysis. Once the haplotype blocks are identified, they can be treated as alleles and tested for LD or association using, for example, multi-allelic TDT or Hendrick's multi-allelic extension of D′ (Spielman, R. S., et al., Am. J. Hum. Genet. 52:506-516 (1993); Lewontin, R. C.; Genetics 49:49-67 (1964); Hedrick, P. W., Genetics 117:331-341 (1987)); thereby providing a test which reflects the underlying genetic variation in the population more accurately than any individual SNP can. The power of the approach can be seen by comparing the noisy single-marker analyses of LD (FIGS. 1A and 1C) with the complementary analyses performed on the underlying haplotype blocks (FIGS. 1B and 1D). The latter analyses show that LD and association decay essentially monotonically, with the decrease occurring in abrupt drops reflecting the sites of significant historical recombination.

The approach is particularly useful for localizing the position of a disease-susceptibility locus. FIGS. 1E and 1F show association analysis with the CD phenotype using either single-marker analysis (1E) or haplotype methods (1F). The latter analysis makes clear that the association with CD is broad and fairly constant across a large region spanning roughly 250 kb. This association reflects the observations of numerous SNPs with the same, significant association to CD as these individual significant results are produced by SNPs with alleles that uniquely identify the long risk haplotype. It is noteworthy that this association peak is not expected to be monotonic in the case of a modest risk factor for reasons described elsewhere for allele sharing among affected siblings at such loci (Kruglyak, L., et al., Am. J. Hum. Genet. 56:1212-23 (1995)). Such a large region with roughly constant association is both encouraging (with respect to the prospects of detecting such ancestral segments) and sobering (both with respect to the prospects of detecting such ancestral segments and with respect to the challenge of fine-structure localization of the disease-causing mutation).

Haplotype blocks are also valuable because they provide a simple method for selecting a subset of SNPs capturing the full information required for population association to find common disease-susceptibility alleles. Once the block structure is defined, it is sufficient to genotype a single SNP to describe block diversity in regions with two haplotypes; two SNPs in regions with three haplotypes; and three SNPs in regions with four haplotypes. Thus, haplotype blocks across the entire 500 kb region can be exhaustively tested with a particular set of 24 SNPs. In fact, considerably fewer SNPs can be utilized by testing every other block, given the strong haplotype correlation among consecutive blocks.

It is important to consider whether the process of SNP selection could have influenced these results. The SNPs studies were ascertained by essentially complete resequencing of 8 individuals. Because the screen included 7 CD patients, it was considered whether this significantly biased the SNP ascertainment. To examine this, a 100 kb region of sequences in the center of the region was selected and the current method of SNP detection was compared to the SNPs identified by the International SNP Map Working Group (ISMWG: The International SNP Map Working Group, Nature 409: 928-33 (2001)). In the screened sequence, the ISMWG reported 54 SNPs and the present study identified 47 of them (86%). This is consistent with the independent observation that about 85% of SNPs identified by the ISMWG in its broad multiethnic panel were found to be polymorphic in a Caucasian panel and suggests that there is no significant bias in the ascertainment of SNPs used here due to ascertainment on primarily affected individuals. In addition, 150 SNPs in this region not reported by the ISMWG were discovered.

The analysis above included only SNPs with minor allele frequency >5%. 6 rarer SNPs that occurred with minor alleles occurring 10-20 times in the data set of >500 chromosomes were not studied. This rare allele fell exclusively or nearly exclusively on one of the major haplotype patterns. This indicates that the rare SNPs have a more recent ancestry and that they contribute to diversity only by creating rare subtypes of common haplotype patterns. Since each individual chromosome has not been sequenced completely, it is likely that additional rare variants are carried on some of the chromosomes sampled. Thus, when limited haplotype diversity is described, complete sequence identity is not implyed, but rather that chromosomes fall into a small number of deep clades. Chromosomes within a clade may differ at one or a few rare SNPs, while chromosomes in different clades differ at many SNPs.

It was noted that SNPs at CpG sites were initially eliminated from the analyses because the higher mutation rate at such sites (Krawczak, M., et al., Am. J. Hum. Genet. 63:474-88 (1998); Nachman, M. W. et al., Genetics 156:297-304 (2000)) might cause recurrent mutations and confound the analysis. Reviewing the 16 high frequency CpG SNPs genotyped in this region, it is noteworthy that 13 have alleles that align perfectly with the haplotype patterns described in FIG. 1. Only 1 of the 16 would add significantly to the overall heterozygosity of the block in which it fell.

The analysis of this region of chromosome 5q31 in a European-derived population indicates that: the region may be largely parsed into discrete blocks of 10-100 kb; that each block has only a few common haplotypes; and that the haplotype correlation between blocks gives rise to long-range linkage disequilibrium.

In several data sets, comprehensive SNP genotyping in small regions (1-5 kb upstream form a candidate gene) has indicated very limited haplotype diversity. Drysdale et. al. genotyped 12 SNPs in a 2 kb promoter stretch of β₂AR, and discovered three haplotypes accounting for 95% of chromosomes in Caucasians with the two most common differing strikingly at 8 to 12 sites (Drysdale, C. M., et. al. PNSA 97:10483-10488 (2000)). Park, et. al. tested 5 SNPs in 2 kb of 5′ untranslated sequence in the thrombin receptor gene and discovered 3 haplotypes which accounted for 91% of all chromosomes in a Korean population (Park, H. Y., et al. Clin Exp Pharmacol Physiol 27: 690-693 (2000)). Jordanides, et. al., examined 4 polymorphisms at the IL6 locus and observed 3 haplotypes accounting for 84% of chromosomes (Jordanides, N., et al, Genes Immun. 1:451-455 (2000)). Similarly, D'Alfonso et. al. typed 11 SNPs in the 3 kb upstream for IL10 and described three major haplotypes (D'Alfonso, S., et al., Genes Immun. 1:231-233 (2000)). Templeton et. al. have undertaken a more detailed examination of variation at LPL (reflecting essentially complete ascertainment of polymorphisms in more than 100 chromosomes) and describe diversity as consisting primarily of a limited number of major clades at each end of the gene separated by an apparent recombinational hotspot (Templeton, A. R., et al., Genetics 156, 1259-1275 (2000)).

Studies of larger regions involving spares SNP maps have also been performed and these results are consistent with the above described observations. Bonnen, et. al., studied 14 SNPs across 150 kb at the ATM locus, but even at this density noted three haplotypes in Caucasians that together accounted for >80% of all chromosomes (Bonnen, P. E. et. al., Am. J. Hum. Genet. 67, 1437-514 (2000)). Moffatt, et. al., described patterns of pairwise LD falling into “distinct islands” suggesting regions of strong LD separated by recombinational hotspots (Moffatt, M. E., et al., Hum. Mol. Genet. 9, 1011-1019 (2000)).

The structure of these blocks and their lack of diversity have considerable implications for human population genetics, since it suggests that models including both recombinational hotspots (reflecting the apparent clustering), and recent bottlenecks (accounting for the lack of haplotype diversity over long distances seen among Caucasians) may be necessary.

The results also define a powerful framework for medical genetic studies. Traditionally, association studies between a disease and a gene have involved testing individual SNPs in and around the gene. This approach suffers from being statistically weak and having no clear endpoint. On the one hand, true association may be missed due to the incomplete information provided by individual SNPs. On the other hand, negative results pertain to the individual SNP tested, but do not rule out association involving other SNPs in the gene (even those in strong marker to marker LD with the SNP tested). In addition, in cases where a positive result is detected, there is no assurance that the detected allele is causal since it may simply be in LD with another polymorphism (potentially several genes away). A more comprehensive approach is to use a sufficiently dense SNP map to define the underlying haplotype blocks across a gene. A subset of SNPs sufficient to uniquely distinguish the major haplotypes in each block can then be selected and associations within each block can be definitively tested. In this manner, it is straightforward to perform an exhaustive test of whether common population variation in a gene is associated with a disease (above a specified level of genotype relative risk and allele frequency for the disease susceptibility allele.)

Finally, the approach provides a precise framework for creating a comprehensive LD map of the human genome for any given population. By testing a sufficiently large collection of SNPs (in the range of 300,000 for the European population), it should be possibly to define all of the underlying haplotype blocks. Once this map is produced, it provides an optimal reference set of SNPs for examination in any subsequent genotyping study.

Particular methods of selecting, detecting, amplifying, genotyping and data checking samples for use in the methods of the invention are described in the Examples of this application. It should be recognized, however, that any suitable methods known to those of skill in the art can be utilized. The following methods are further examples of methods that can be so utilized.

Analysis of SNPs

A. Preparation of Samples

SNPs are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed. For example, if the target nucleic acid is a cytochrome P450, the liver is a suitable source.

Many of the methods described below require amplification of DNA from target samples. This can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19:4967 (1991); Eckert et al., PCR Methods and Applications 1, 17 (1991); PCR (eds. McPherson et al., IRL Press, Oxford); and U.S. Pat. No. 4,683,202 (each of which is incorporated by reference for all purposes).

Other suitable amplification methods include the ligase chain reaction (LCR) (see Wu and Wallace, Genomics 4:560 (1989); Landegren et al., Science 241:1077 (1988)), transcription amplification (Kwoh et al., Proc. Natl. Acad. Sci. USA 86:1173 (1989)), and self-sustained sequence replication (Guatelli et al., Proc. Nat. Acad. Sci. USA 87:1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

B. Detection of SNPs in Target DNA

There are two distinct types of analysis depending whether an SNP in question has already been characterized. The first type of analysis is sometimes referred to as de novo characterization. This analysis compares target sequences in different individuals to identify points of variation, i.e., polymorphic sites. By analyzing a group of individuals representing the greatest variety patterns characteristic of the most common alleles/haplotypes of the locus can be identified, and the frequencies of such populations in the population determined. Additional allelic frequencies can be determined for subpopulations characterized by criteria such as geography, race, or gender. The second type of analysis is determining which form(s) of a characterized polymorphism are present in individuals under test. There are a variety of suitable procedures, which are discussed in turn.

1. Allele-Specific Probes

The design and use of allele-specific probes for analyzing SMPs is described by e.g., Saiki et al., Nature 324:163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15 mer at the 7 position; in a 16 mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

2. Tiling Arrays

The SNPs can also be identified by hybridization to nucleic acid arrays. Subarrays that are optimized for detection of a variant forms of a precharacterized polymorphism can also be utilized. Such a subarray contains probes designed to be complementary to a second reference sequence, which is an allelic variant of the first reference sequence. The inclusion of a second group (or further groups) can be particular useful for analyzing short subsequences of the primary reference sequence in which multiple mutations are expected to occur within a short distance commensurate with the length of the probes (i.e., two or more mutations within 9 to 21 bases).

3. Allele-Specific Primers

An allele-specific primer hybridizes to a site on target DNA overlapping a SNP and only primes amplification of an allelic form to which the primer exhibits perfect complementarily. See Gibbs, Nucleic Acid Res. 17, 2427-2448 (1989). This primer is used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. The method works best when the mismatch is included in the 3′-most position of the oligonucleotide aligned with the polymorphism because this position is most destabilizing to elongation from the primer.

4. Direct-Sequencing

The direct analysis of the sequence of any samples for use with the present invention can be accomplished using either the dideoxy-chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

5. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

6. Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target sequences.

7. Single Base Extension

An alternative method for identifying and analyzing SNPs is based on single-base extension (SBE) of a fluorescently-labeled primer coupled with fluorescence resonance energy transfer (FRET) between the label of the added base and the label of the primer. Typically, the method, such as that described by Chen et al., (PNAS 94:10756-61 (1997)), uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently-labeled dideoxyribonucleotides (ddNTPs) in dye-terminator sequencing the effect of mtDNA D-loop sequence polymorphism on milk production, each cow was the next generation of the herd.

The genomic maps and the methods of the invention can be readily used in several ways. The mapping of discrete haplotype regions which are at most minimally recombinagenic permits, for example, the subsequent identification of genotypes and phenotypes associated with particular blocks, the localization of the position of a disease-susceptibility locus of a disease as well as the development of diagnostic assays for disease phenotypes.

For example, linkage studies can be performed for particular haplotypes in the discrete haplotype blocks because such haplotypes contain particular linked combinations of alleles at particular marker sites. A marker can be, for example, a RFLP, an STR, a VNTR or a single nucleotide as in the case of SNPs. Since the block has been identified as being primarily nonrecombinagenic, the detection of a particular marker will be indicative of a particular haplotype. If, through linkage analysis, it is determined that a particular haplotype is associated with, for example, a particular disease phenotype, then the detection of the marker in a sample derived from a patient will be indicative of an increased risk for the particular disease phenotype. Additionally, if a particular phenotype is known to be associated with a particular discrete haplotype block, then the block can be sequenced and scanned for coding regions that code for products that lead to the disease phenotype. In other words, the position of a disease-susceptibility locus of a disease can be located. In this way, a map of the invention comprising discrete haplotype blocks and sites of recombination can be used to identify the location of a gene or genes responsible for producing particular diseases.

Linkage analysis can be performed by identifying genetic markers in the discrete haplotype blocks. For example, after a block has been mapped, it can be screened for genetic markers, e.g., polymorphic sites, e.g., SNPs, that, in a given population, can have different sequences at a particular site. The presence of these sequence differences means that there are different versions or “alleles” that are possible at a polymorphic site. For example, in the case where the marker involves a single nucleotide, the marker is a single nucleotide polymorphic site at which different alleles might be possible in a population. If, for example, two samples are sequenced and it is determined that, at a particular site in the block, one sample had an adenine present and the other sample had a thymine present, then it can be concluded that the site is a polymorphic site with at least two possible allelic variations possible, namely, an “A” allele or a “T” allele. Once a marker, e.g., SNP, has been identified in a discrete haplotype block, linkage analysis can be performed.

Linkage analysis can be accomplished, for example, by taking samples from individuals from a particular population and determining which allelic variants the individuals have at the marker sites. Using algorithms known in the art, the occurrence of a particular allele can be compared to, for example, a particular phenotype in the population. If, for example, it is found that a high proportion of the population that has a particular disease phenotype also carries a particular allele at a particular polymorphic site—then one can conclude that the particular allele is linked to the particular phenotype in that population. Additionally, since the markers were identified in discrete haplotype blocks, the particular allele will be indicative for a haplotype that spans the entire discrete haplotype block. The marker allele is, therefore, determined to be linked to a particular phenotype and also linked to a particular haplotype that spans the discrete haplotype block. Thus, by identifying genetic markers, e.g., SNPs, in discrete haplotype blocks, linkage analysis can be performed that allows for the conclusion that a particular phenotype is linked to a particular haplotype that spans then entire discrete haplotype block.

All publications, patents, patent applications and information from databases cited above are hereby incorporated by reference in their entirety for all purposes to the same extent as if each individual publication or patent application were specifically and individually indicated to be so incorporated by reference.

The invention is now further described in the following non-limiting examples.

### EXEMPLIFICATION

### EXAMPLE 1

Identifying Genetic Variation in the 5q31 Gene Cluster for Susceptibility to Crohn Disease

Linkage disequilibrium (LD) mapping provides a powerful method for fine-structure localization of disease susceptibility genes. It has been widely applied to study rare monogenic traits, but has been not yet been widely applied to common disease (Horikawa, Y., et al., Nat. Genet. 26:163-175 (2000)). A systematic approach for LD mapping was designed and applied to localize a gene (IBD5) conferring susceptibility to a form of inflammatory bowel disease (IBD). Using linkage analysis, the IBD5 locus had been previously mapped, primarily in families with early onset cases, to a large region spanning 18 cM of chromosome 5q31 containing the cytokine gene cluster (p<10⁻⁴). Using dense genetic maps of microsatellite markers and single nucleotide polymorphisms (SNPs), it was found that IBD5 is contained within a common haplotype spanning about 250 kb that shows strong association with the disease (p<2×10⁻⁷). The precise disease-causing mutation cannot be readily identified from the genetic data alone, because strong LD across the region results in multiple SNPs with evidence consistent with that expected for the IBD5 locus. The results have important implications for the genetic basis of IBD in particular and for LD mapping in general.

The majority of IBD patients can be classified as having Crohn Disease (CD) or ulcerative colitis (UC). The incidence of CD and UC in most Western countries is 5-10 cases per 100,000 inhabitants, resulting in an estimated one million IBD patients in the United States alone. CD and UC are idiopathic inflammatory diseases of the bowel associated with distinct clinical and pathological profiles. Specifically, CD is characterized by discontinuous, transmural inflammation potentially involving any part of the gastrointestinal tract, whereas in UC the inflammatory process is continuous and restricted to the mucosa of the large intestine.

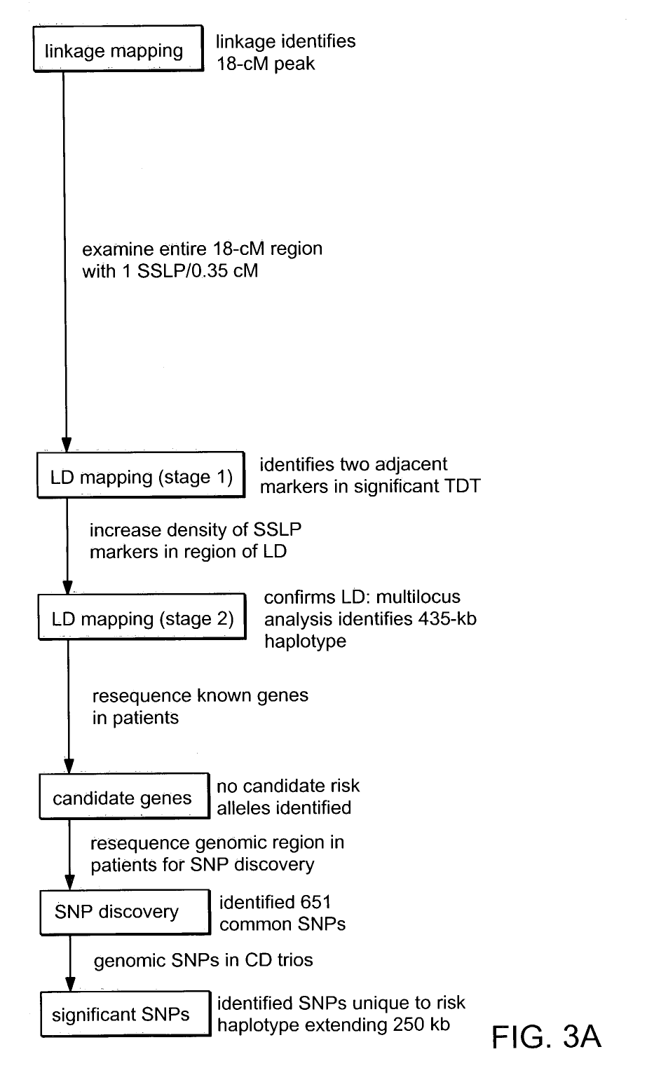

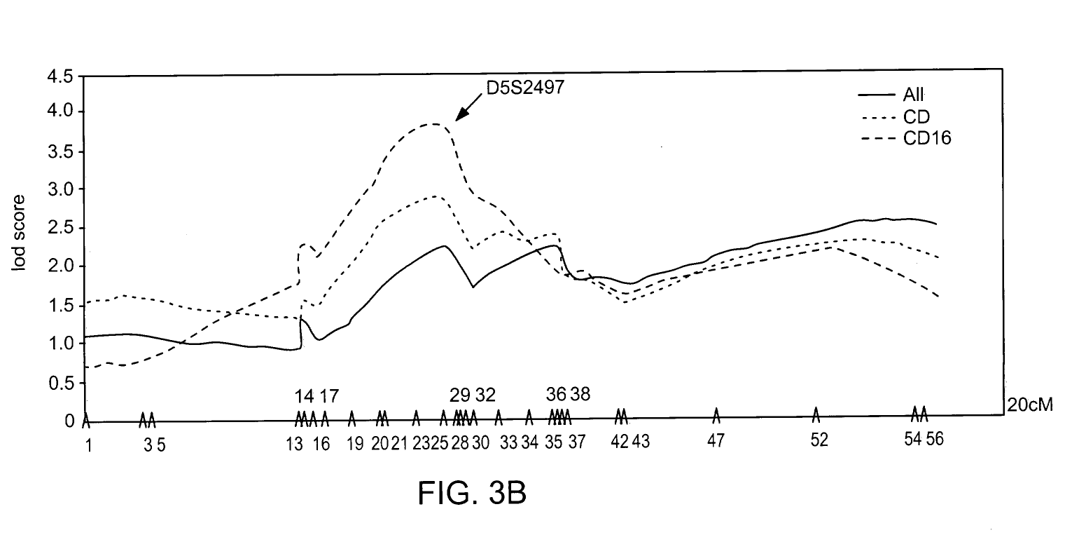

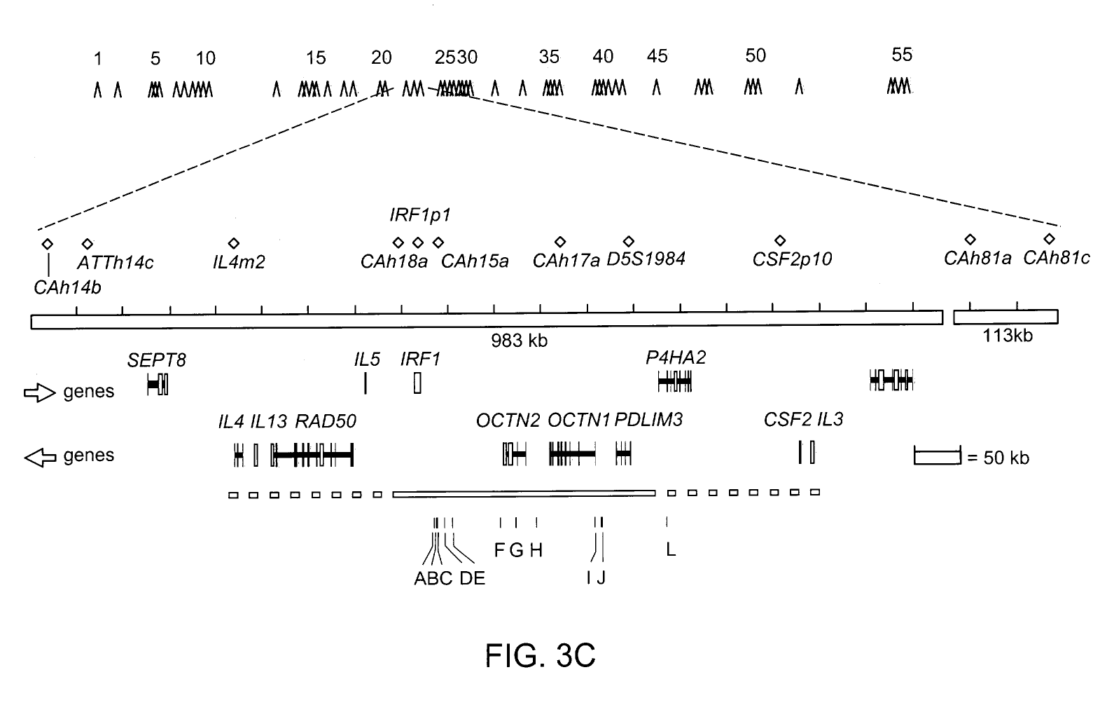

A linkage analysis of 158 nuclear families (referred to as “set A” in Table 1) with multiple siblings affected with IBD was recently reported, providing strong evidence (LOD 3.0) for a gene (IBD5) conferring susceptibility to CD in chromosome 5q31 (Rioux, J. D., et al., Am J Hum Genet 66:1862-1870 (2000)). Linkage was strongest among families containing early-onset cases (i.e., families in which there was at least one affected sibling with an age or diagnosis ≦16) with a LOD of 3.9. The IBD5 gene mapped to the 18 cM region centered at the marker D5S2497 (at which the LOD score is highest) and bounded by the D5S1435 and D5S1480 (at which the LOD scores falls by two units, corresponding to a 100-fold decrease in relative likelihood) (FIG. 3).

With the aim of identifying the IBD5 gene, further studies to more narrowly circumscribe the region by using hierarchical LD mapping were conducted. The LD mapping was performed by using the transmission disequilibrium test (TDT) (Spielman, R. S., et al., Am. J. Hum. Genet. 59, 983-989 (1996)) to identify alleles or haplotypes that are transmitted at an unusually high frequency from heterozygous parents to affected offspring. The analysis was hierarchical in that the density of the genetic map was progressively increased in the region of interest.

A set of 256 Caucasian father-mother-child trios (where the child had CD and at least one parent was unaffected) from the Toronto region, a large Canadian metropolitan area populated by nearly 5 million people with the majority being of European descent; these samples will be referred to as ‘set B’ were originally studied. Approximately half of these trios were taken from the original linkage families (with only one trio per family), while half were from new families (Table 1).

TABLE 1
Summary of trios used in the SSLP and SNP genotyping

| | Number of | | | | |
|---|---|---|---|---|---|
| | pedigrees/ | Genotyping | | Age of diagnosis | |
| | trios | performed | Population | Average; median | Description of Pedigrees |
| SET | 122 CD | 56 | Toronto | 17.2; 18.0 | 50/122 linkage families hadan |
| A | pedigrees¹ | SSLPs | | | affected offspring with age of |
| | | | | | diagnosis ≦ 16 |
| SET | 256 CD | 64 | Toronto | 18.4; 16.0 | 124 trios from original linkage |
| B | trios | SSLPs | | | families (set A), with only one trio |
| | | | | | per family to ensure independence |
| | | | | | for LD analysis |
| | | | | | 132 new trios (not in set A) |
| SET | 139 CD trios | 301 | Toronto | 16.3; | 139 trios consisting of two subsets: |
| C | | SNPs | | subset C1 15.4; | subset Cl - 63 trios from Set B |
| | | | | 14.0 | (18/63 from original linkage |
| | | | | subset C2 17.2; | families) |
| | | | | 16.0 | subset C2 - 76 new trios (not in Set |
| | | | | | A or B) |
| SET | 88 CD | 12 | Quebec | 23.0; 21.5 | 88 trios independently collected for |
| D | trios | significant | | | current study |
| | | SNPs² | | | |

¹as described in reference 2
²set of 12 significant SNPs as described in Table 5

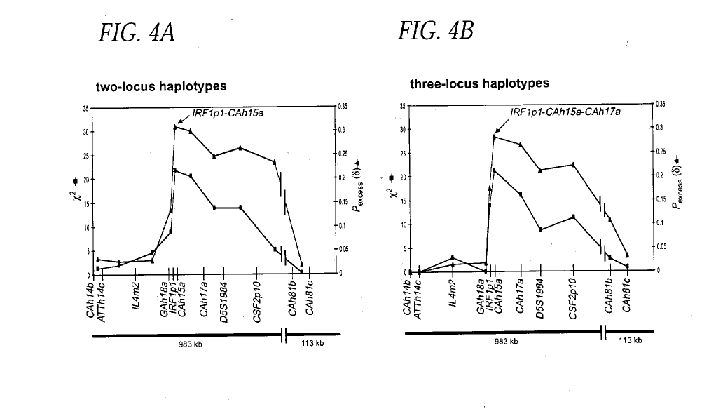

Importantly this collection of trios had children affected at an early age (average and median age of diagnosis of 18.4 and 16.0 years, respectively) as compared to the “classic” age distribution of onset with a majority of cases diagnosed in the third and fourth decades of life (e.g., average and median age of diagnosis of >33 and 29 years, respectively) (Loftus, W. V., et al., Gastroenterology 114: 1161-1168 (1998)). These samples were genotyped for 56 microsatellite polymorphisms distributed throughout the region, with average distance between markers of about 0.35 cM. Each allele of each marker was examined for evidence of transmission disequilibrium. Significant TDT results (p<0.001) were found for two of the 56 loci (Table 2A, FIG. 4). The two loci were IRF1p1 (allele 156, X²=14.2, p=0.00016) and D5S1984 (allele 222, X²=12.6, p=0.00039). Notably, the two loci were adjacent to one another and were within 1 cM of the marker D5S2497, at which the peak linkage score had been obtained. To assess the significance of these results, permutation tests were performed in which the genotype data was held constant but the transmission status of each chromosome (transmitted vs. untransmitted) was assigned at random. In 10⁶ permutations of the entire data set of 56 markers, a single-allele X² value >14.2 was observed only 15796 times (corresponding to an empirical p-value of 0.016) and only 911 simulations had 2 markers with X²>12.5 (corresponding to an empirical p-value of 0.00091 corrected for multiple testing). Because the TDT tests of association are completely independent of our previously reported linkage result, they provide strong confirmation of the presence of a susceptibility gene for CD. The risk alleles at the two markers showed a transmission ratio (defined as the ratio of cases in which the allele was present on the transmitted (T) versus untransmitted (U) chromosome in a heterozygous parent) of 1.8:1 and 1.9:1.

TABLE 2A
Summary of the first stage of LD mapping using mierosatellite markers.

| | | Source | Estimated | Distance | Previous | | | | |
|---|---|---|---|---|---|---|---|---|---|
| Marker | Marker | of | Genetic | to next | linkage | TDT results⁴ | | | |
| # | Name | Marker¹ | Position¹ | marker | results (MLOD)³ | Allele | T:U | X² | pvalue |
| 1 | D5S1435 | G | 128.50 | 0.50 | 0.76 | 115 | 1:8 | 8.49 | 0.004 |
| 2 | AFMa113ye9 | G | 129.00 | 0.83 | | — | | — | — |
| 3 | D5S1505 | M | 129.83 | 0.00 | 0.79 | — | 1:6 | 4.15 | 0.042 |
| 4 | D5S1384 | U | 129.83 | 0.00 | | — | | — | — |
| 5 | D5S471 | G | 129.83 | 0.57 | 0.79 | 238 | 1.8 | 8.18 | 0.0042 |
| 6 | D5S632 | G | 130.40 | 0.20 | | — | | — | — |
| 7 | D5S818 | M | 130.60 | 0.20 | | — | | — | — |
| 8 | D5S2502 | M | 130.80 | 0.10 | | — | | — | — |
| 9 | AFMB352XH5 | G | 130.90 | 0.04 | | — | | — | — |
| 10 | D5S1975 | G | 130.94 | 0.00 | | — | | — | — |
| 11 | D5S622 | G | 130.94 | 1.86 | | 186 | 1.7 | 4.95 | 0.026 |
| 12 | D5S2059 | G | 132.80 | 0.85 | | 190 | 2.2 | 4.17 | 0.041 |
| 13 | D5S615 | U | 133.65 | 0.00 | 1.8 | — | | — | — |
| 14 | D5S804 | M | 133.65 | 0.00 | 1.8 | — | | — | — |
| 15 | D5S1495 | M | 133.65 | 0.00 | 1.8 | 382 | 1.5 | 4.37 | 0.037 |
| 16 | GATA68A03 | M | 133.65 | 0.35 | 2.2 | — | | — | — |
| 17 | D5S809 | M | 134.00 | 0.40 | 2.1 | — | | — | — |
| 18 | D5S2120 | G | 134.40 | 0.20 | | — | | — | — |
| 19 | D5S642 | G | 134.60 | 0.65 | 2.6 | — | | — | — |
| 20 | D5S2057 | G | 135.25 | 0.00 | 3.1 | — | | — | — |
| 21 | D6S2110 | G | 135.25 | 0.62 | 3.1 | — | | — | — |
| 22 | IRF1p1 | S | 135.87 | 0.19 | | 156 | 1.9 | 14.25 | 0.00016 |
| 23 | D5S1984 | G | 136.06 | 0.16 | | 222 | 1.8 | 12.57 | 0.00039 |
| 24 | CSF2p10 | S | 136.22 | 0.58 | | 307 | 1.5 | 4.52 | 0.033 |
| 25 | D5S2497 | G | 136.80 | 0.10 | 3.9 | 129 | 1.9 | 5.45 | 0.020 |
| 26 | w/2492/230wa7 | G | 136.90 | 0.10 | | — | | — | — |
| 27 | W866/057vg5 | G | 137.00 | 0.10 | | — | | — | — |
| 28 | D5S1766 | U | 137.10 | 0.10 | 3.5 | 245 | 2.1 | 5.90 | 0.015 |
| 29 | D5S808 | M | 137.20 | 0.10 | 3.3 | — | | — | — |
| 30 | D5S458 | G | 137.30 | 0.00 | 3.1 | — | | — | — |
| 31 | D5S396 | G | 137.30 | 0.09 | | — | | — | — |
| 32 | D5S2053 | G | 137.39 | 0.56 | 3.0 | — | | — | — |
| 33 | D5S1995 | G | 137.95 | 0.69 | 2.8 | — | | — | — |
| 34 | D5S2115 | G | 138.64 | 0.68 | 2.4 | — | | — | — |
| 35 | IL9 | M | 139.32 | 0.01 | 2.0 | — | | — | — |
| 36 | D5S816 | M | 139.33 | 0.07 | 2.0 | — | | — | — |
| 37 | D5S393 | G | 139.40 | 0.10 | 2.0 | — | | — | — |
| 38 | D5S399 | G | 139.50 | 0.90 | 2.0 | — | | — | — |
| 39 | D5S479 | G | 140.40 | 0.10 | | — | | — | — |
| 40 | AFM350UB1 | G | 140.50 | 0.10 | | — | | — | — |
| 41 | D5S1983 | G | 140.60 | 0.12 | | 116 | 1.6 | 5.69 | 0.017 |
| 42 | D5S476 | G | 140.72 | 0.00 | 1.7 | — | | — | — |
| 43 | D5S500 | G | 140.72 | 0.28 | 1.7 | — | | — | — |
| 44 | AFMB290YC9 | G | 141.00 | 0.82 | | — | | — | — |
| 45 | D5S414 | G | 141.82 | 0.98 | | — | | — | — |
| 46 | D5S2009 | G | 142.80 | 0.12 | | 140 | 2.0 | 8.12 | 0.004 |
| 47 | D5S658 | G | 142.92 | 0.00 | 2.0 | — | | — | — |
| 48 | D5S2116 | G | 142.92 | 1.08 | | — | | — | — |
| 49 | D5S2011 | G | 144.00 | 0.06 | | — | | — | — |
| 50 | D5S2119 | G | 144.06 | 0.00 | | — | | — | — |
| 51 | D5S1979 | G | 144.06 | 1.15 | | — | | — | — |
| 52 | D5S2017 | G | 145.21 | 2.19 | 2.2 | 91 | 1.7 | 4.27 | 0.039 |
| 53 | D5S2859 | M | 147.40 | 0.09 | | — | | — | — |
| 54 | D5S436 | G | 147.49 | 0.00 | 1.6 | — | | — | — |
| 55 | D5S207 | M | 147.49 | 0.00 | | — | | — | — |
| 56 | D5S1480 | M | 147.49 | | 1.6 | — | | — | — |

¹Abbreviations: G, Genethon; M, Marshfields; U, Utah; S, Designed by authors from genomic sequence.
²Estimated from genetic (Genethos, Marshfield) and physical (data not shown) map information
³Linkage data for the CD subgroup with early onset disease as seen in FIG. 1 and reference 12
⁴Results are shown only if pvalue < 0.05

Having found evidence of LD, the next step was to study a denser collection of markers in the implicated region to confirm the results. (Had no evidence of LD been found, the next step would have been to double the density of markers throughout the 18 cM region.) For this purpose, new microsatellite markers were developed from the 680 kb of DNA sequence that was then available for this region (Sved, J. A., Theor. Pop. Biol. 2:125-141 (1971)). Eight of these markers amplified well, were polymorphic when tested on the DNA samples, and were used to genotype the 256 trios in set A (Table 2B and FIG. 1). The marker with the most significant TDT result was CAh17a (X²=13.8, p=0.0002) and was located between the previous two markers with significant scores; the immediately flanking markers showed weaker evidence of LD.

TABLE 2B
Summary of Combined LD Mapping Information.

| | | Distance | | | | | |
|---|---|---|---|---|---|---|---|
| | | to next | LD | | | | |
| Marker | Marker | marker | mapping | TDT results | | | |
| # | Name | (kb) | stage | Allele | T:U | X² | pvalue |
| 57 | CAh14b | 43 | 2 | — | | — | — |
| 58 | ATTh14e | 167 | 2 | — | | — | — |
| 59 | IL4m2 | 164 | 2 | — | — | — | — |
| 60 | Gah18a | 21 | 2 | 193 | 1.5 | 5.70 | 0.017 |
| 22 | IRFF1p1 | 24 | 1 | 156 | 1.9 | 14.25 | 0.00016 |
| 61 | CAh15a | 130 | 2 | 373 | 1.5 | 4.0 | 0.045 |
| 62 | CAh17a | 97 | 2 | 140 | 1.7 | 13.80 | 0.00020 |
| 23 | D5S1984 | 163 | 1 | 222 | 1.8 | 12.57 | 0.00039 |
| 24 | CSF2p10 | N.D. | 1 | 307 | 1.5 | 4.52 | 0.033 |
| 63 | CAh81b | 85 | 2 | — | | — | — |
| 64 | Cah81c | | 2 | — | | — | — |

The boundaries of the implicated region were then defined, by using multi-locus methods to identify the underlying haplotypes conferring susceptibility. Haplotype analysis can provide greater specificity than single-marker analysis in the identification of risk-conferring chromosomes, thereby revealing a higher transmission ratio. In principle, a sufficiently dense haplotype should uniquely mark susceptibility chromosomes.

Analysis of two- and three marker haplotypes indeed increased the strength of the evidence (FIG. 4), showing significant TDT and P_{excess} results even around loci for which the single-marker TDT results were not significant and increasing the transmission ratio to greater than 2:1. Strong evidence of uninterrupted LD was found across a ˜400 kb region from Gah18a to CSF2p10, with the region near IRF1p1-CAh15a-CAh17a showing the strongest evidence (with normal p-values in the range of 3×10⁻⁶; See FIG. 2). The risk haplotype can be defined by the six markers GAh18a-IRF1p1-Cah15a-Cah17a-D5S1984-CSF2p10 (alleles 193-156-373-140-222-307).

Since the LD analysis indicated that the IBD5 gene lies within the ˜400 kb region delimited by Gah18a and CSF2p10, the genes therein were examined with respect to their potential role in the pathophysiology of IBD. Expanding the search to contain the genomic region from IL4 to IL3 (considering the potential interest of these cytokine genes), 11 known genes were identified (FIG. 3). Although the precise etiology of IBD is unknown, it is becoming clear that the chronic inflammation in the gastrointestinal tract is at least partly due to interaction between the host's immune system and the enteric microflora normally present across the mucosal wall. Initiation or maintenance of the chronic inflammatory processes may be due to disturbance of the normal balance of the local immune regulatory mechanisms, the local flora, or the mucosal wall's integrity. Strikingly, five of the known genes in this small region are biologically plausible candidates for a CD susceptibility locus. There are three genes encoding immunoregulatory cytokines (IL-4, IL-13 and IL5). Different patterns of expression of these three cytokines have been observed between early and chronic lesions, and between CD and UC lesions. It is believed that these cytokine profiles reflect differences in the T_{H}1/T_{H}2 T cell balance that may play an important pathophysiological role. The region also contains the gene for interferon regulatory factor-1 (IRF-1), a transcription factor that has been shown to be important in T_{H}1/T_{H}2 T cell balance, to be regulated by immunoregulatory and proinflammatory cytokines, and to play a major role in the specific mucosal immune defense mechanisms. Moreover, IRF-1 expression appears to be increased in mucosal mononuclear cells of CD patients when compared to UC patients or healthy controls (Clavell, M. et al., J. Pediatr. Gastroenterol. Nutr. 30:43-47 (2000)). Finally, the region also contains the prolyl 4-hydroxylase alpha II (P4HA2) gene. Although unrelated to mucosal immunity, it is an interesting candidate for a CD susceptibility gene because prolyl 4-hydroxylase activity is significantly greater in the mucosa of CD patients (Farthing, M. F., et al., Gut 19, 743-747 (1978). Moreover, this enzyme is essential to collagen synthesis, is potentially important for mucosal wall integrity, and may contribute to the fibrosis which is characteristic of CD.

In addition to the identification of the known genes, this genomic region was also examined for the presence of unidentified genes by using the GENSCAN gene-prediction software and by searching for EST clusters using BLAST alignment. Eight predicted genes and four EST clusters were identified that did not overlap with the previously known genes and only one of the predicted genes had overlap with an EST cluster. From the comparison of the known mouse and human sequence, 47 conserved non-coding sequences were reported in this region (Loots, G. G., et al., Science 2888:136-140 (2000). Potential mutations in all of the 11 genes were searched for by resequencing the complete transcribed regions (5′UTR, coding, and 3′UTR) as well as 1 kb upstream of the transcription start site. A total of 16 SNPs were identified in these 11 genes (Table 3), but there were no obvious candidate mutations. Only two of the SNPs showed nominally significant TDT results: a silent substitution in the coding region of the OCTN2 gene (SNP-IGR2222a_{—}2 with X²=8.2, p=0.004) and a missense substitution (Thr→Ile) in the OCTN1 gene (SNP-IGR3016a_{—}1 with X²=8.9, p<0.003). The two genes encode structurally similar organic cation transporters. OCTN2 is a specific carnitine transporter, while OCTN1 is a multi-specific cation transporter of unknown function (Burckhardt, G., et al., Am. J Phsiol. Renal. Physiol. 278:F853-F866 (2000)). Neither SNP appears to be a strong candidates for IBD5: the first is a silent substitution; the second seems unlikely to have a severe effect on the protein, inasmuch as isoleucine is found in the analogous position in mouse OcTN1 and in mouse, rat and human OCTN2 (Burckhardt, G., et al., Am. J. Phsiol. Renal. Physiol. 278:F853-F866 (2000)). Moreover, subsequent analysis of the region (see below) turned up SNPs with stronger evidence of transmission disequilibrium with the CD phenotype and showed that these SNPs were not unique to the risk haplotype.

Because no obvious candidates emerged from analysis of the transcribed regions of the known genes, a comprehensive SNP-based analysis of the region was undertaken to obtain a precise description of the properties and extent of the risk haplotype. A reference sequence of 984 kb (containing all of the sequence from CAh14b to CSF2p10) was assembled by combining the sequence reported by Frazer et al (Frazer, K. A., et al, Genome Res 7495-7512 (1997)). Systematic SNP discovery was then undertaken by direct resequencing of PCR-products from eight individuals, consisting of seven CD patients and one control. The seven CD patients were selected to maximize the chance that they carried mutations at the IBD5 locus. Specifically, the CD samples were taken from families showing linkage to chromosome 5q31. They were also selected based on whether they carried the risk haplotype at the six markers from GAh18a through CSF2p10 (see above). One individual was homozygous for the risk haplotype, three were heterozygous, and three did not appear to carry this haplotype. This set of samples reflected the diversity observed in the entire dataset and was chosen to ensure the identification of the risk allele as well as SNPs in the overall sample collection.

TABLE 3
Summary of SNPs Located Within the Transcribed Regions of Known Genes
in the Region of LD.

| Gene ID | LocusLink ID¹ | SNP ID | Position² | Location | Comment |
|---|---|---|---|---|---|
| IL4 | 3565 | IGR1029a_1 | 230.0 | Exon 1 | 5′UTR |
| IL13 | 3596 | IGR1055a_1 | 243.0 | Exon 4 | 3′UTR |
| | | IGR1056a_2 | 243.0 | Exon 4 | 3′UTR |
| | | IGR1046a_3 | 243.0 | Exon 4 | 3′UTR |
| | | IGR1057a_1 | 243.5 | Exon 4 | R129Q |
| RAD50 | 10111 | IGR1092a_1 | 261.0 | Exon 25 | 3′UTR |
| IL5 | 3567 | no SNPs | n/a | n/a | |
| IRF1 | 3659 | IGR2011b_1 | 413.0 | Exon 1 | 5′UTR |
| | | IGR2011b_2 | 413.5 | Exon 1 | 5′UTR |
| | | IGR2020a_2 | 417.5 | Exon 7 | silent |
| | | IGR2026a_2 | 420.5 | Exon 10 | 3′UTR |
| OCTN2 | 6584 | IGR2222a_2 | 518.5 | Exon 4 | silent |
| OCTN1 | 6583 | IGR3002a_1 | 569.0 | Exon 7 | silent |
| | | IGR3016a_1 | 576.5 | Exon 5 | T306I |
| RIL | 8572 | IGR3138a_1 | 637.0 | Exon 3 | silent |
| P4AH2 | 8974 | no SNPs | n/a | n/a | |
| CSF2 | 1437 | CSFex4_6632 | 828.0 | Exon 4 | I73T |
| IL3 | 3562 | IL3_4400 | 843.0 | Exon 1 | S27P |

¹see the worldwide web at .ncbi.nlm.nih.gov/LocusLink/
²Approximate physical position (kb) on the 983 kb reference sequence.

The SNP discovery effort was divided into three regions (Table 4): a “core” region of 285 kb (Gah18a to D5S1984) in which peak association was observed, together with a “proximal” region of 150 kb (IL4 to GAh18a) and a “distal” region of 200 kb (D5S1984 to IL3). In the core region, 94% of the region was successfully resequenced. In the proximal region, sequencing assays were primarily designed to cover exons of known genes and regions with significant homology (>100 bp with >80% identity) to the known mouse sequence syntenic to this region (total of 100 kb resequenced). In the distal region there was no known mouse sequence, so assays were designed to cover exons of known genes as well as assays to cover every other 500 bp segment (total of 85 kb of reference). In all, 952 different PCR assays in each of the eight DNA samples were examined, with an overall success rate of about 94%. A total of 651 candidate SNP2 were discovered in these three regions, as summarized in Table 4.

TABLE 4
Summary of SNPs Discovered and Genotyped
in the Region of LD Surrounding IBD5

| | | #SNPs | #SNPs |
|---|---|---|---|
| Region | Sequence targeted | discovered | genotyped¹ |
| Core | 285 kb contiguous sequence | 472 | 216 |
| | from GAh18a to D5S1984 | | |
| Proximal | 100 kb of noncontiguous sequence | 118 | 24 |
| | over a 150 kb region from | | |
| | IL4 to GAhl8a | | |
| Distal | 85 kb of noncontiguous sequence | 61 | 61 |
| | over a 200 kb region from | | |
| | D5S1984 to IL3 | | |
| | TOTAL = | 651 | 301 |

¹SNPs were considered genotyped of >50 trios were fully genotyped (ie markers with approximately >80% genotype efficiency)

A large proportion of these SNPs were then genotyped to define the risk haplotype and to search for candidates for the IBD5 mutation. Genotyping was performed on set C consisting of 139 trios (Table 1). Sixty-three of these trios (set C1) were taken from set B (which had been analyzed with respect to the SSLP markers) and the remaining 76 (set C2) were newly collected samples. Set C1 thus allows for integration between the SSLP based and SNP-based haplotype data, and set C2 provides an independent test of association, respectively. As before, the patients in set C had early age of onset (average and median age of diagnosis of 16.3 and 15.0 years, respectively). 301 of the SNPs have been genotyped to date (Table 4), resulting in an average marker spacing of about 1.3, 6.2, and 3.3 kb for the core, proximal and distal regions, respectively.

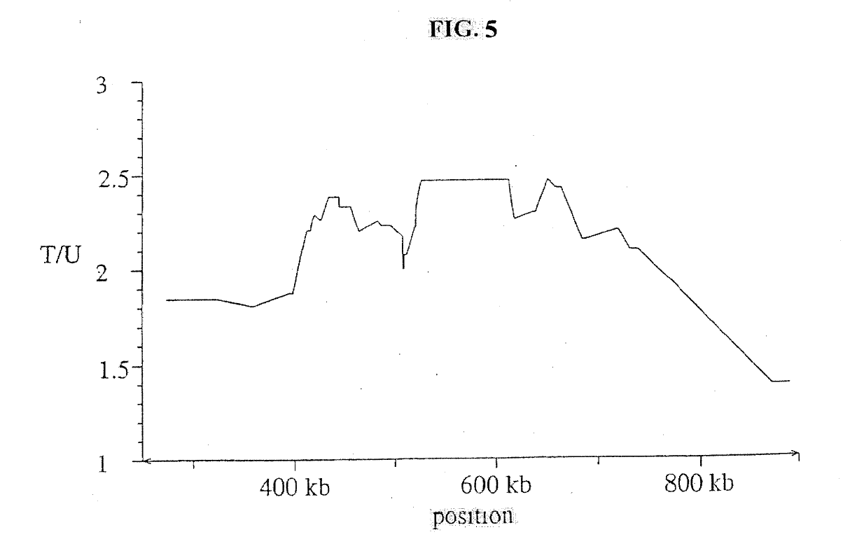

Using this ultra-high density SNP map, it is possible to perform a fine-structure haplotype analysis of the region. A number of new analytical techniques for parsing regions into ancestral segments and for mapping properties such as the transmission ratio are utilized here. These analyses demonstrated that the region can be parsed into haplotype blocks, each in which there existed limited diversity (2 to 4 haplotypes account for >90% of all chromosomes in all segments) and that there is extensive LD across the entire region examined. Multi-locus analyses clearly defined a single risk haplotype showing a maximal transmission ratio of 2.5:1 (FIG. 5). For the samples in set C1, chromosomes bearing the SNP-base risk haplotype corresponded nearly perfectly with those bearing the microsatellite-based risk haplotype (although the SNPs provide much higher resolution). For the samples in set C2, the TDT analysis provides a further independent confirmation of the presence of a susceptibility gene within the region (X²=13.8; p<0.002).

Additional confirmation of the LD observations were provided with an independent dataset derived from a different population. For this purpose, an additional 88 CD trios (set D) from within the Canadian province of Quebec were collected and genotyped with the 12 SNPs which uniquely identify the risk haplotype. All of the same alleles were observed as over-transmitted in this independent dataset and it was further noted that the risk haplotype had a transmitted:untransmitted (T:U) ratio of 1.75 (X²=4.1, p=0.043). These TDT results constitute yet another independent replication of the findings of a CD risk haplotype. The T:U ratio is somewhat smaller than in sets B and C2, although the difference is not statistically different. It is possible however, that the slightly lower T:U ratio may reflect the fact that set D has a somewhat older age of onset (average and median age of diagnosis of 23.0 and 21.5, respectively) or it may be due to the different population histories of Toronto (primarily mixed European) and Quebec (primarily French Canadian). As a final step in proving the association, generalized transmission ratio distortion (TRD) for the haplotype ndependent of CD was ruled out by examining 31 independent trios from 19 reference families from the Centre d'Etude du Polymorphisme Humain (CEPH) panel. There was no evidence that the haplotype was over-transmitted from heterozygous parents in these control trios.

It was next determined whether the properties of the risk haplotype were adequate to explain the observed genetic properties of IBD5. The risk haplotype has the following properties: (1) its frequency among untransmitted chromosomes is 37%, (2) the transmission ratio from heterozygous parents to CD patients is 2.5:1, and (3) the proportion of homozygotes to heterozygotes among affected individuals is 1:1. From these characteristics one can infer the genetic properties of a CD locus carried on such a haplotype. Specifically, the best fit is a model in which one copy of the risk chromosome increases the risk to CD by 2-fold and 2 copies increases the risk to CD by 6-fold. Whether such a CD-susceptibility locus could have given rise to the observed linkage data (corresponding to an MLS of 3 for CD-only families) was also tested. Simulation tests demonstrated that this model would give rise to an MLS>3 in approximately 7% of cases and to an MLS>2 in 20% of cases. This indicated that this haplotype is indeed consistent with the linkage results, although it suggests that the original linkage result (MLS˜3) may reflect some enrichment for families linked to this locus and may have slightly overestimated the effect of the IBD5 locus. This is consistent with the observation that the 5q31 region has not been reported as having a significant LOD score in other genomewide searches, but has previously been seen with a suggestive LOD score in two reports (Cho, J. H. et al., Proc. Natl. Acad. Sci USA 95:7502-7507 (1998); Ma, Y. et al., Inflamm. Bowel Dis. 5:271-278 (1999)). Broadly speaking, the risk haplotype has the properties expected of the IBD5 locus. Moreover, since all of the other common haplotypes in this region are undertransmitted, the causative allele must be unique to the risk haplotype.

Based on the results described above, it can be conclude that the IBD5 mutation/mutations must be located within the 250 kb identified by this LD approach and must be unique to this risk haplotype. In fact multiple SNPs meet this criteria. Of 301 SNPs genotype to date (including all SNPs in known or predicted genes), 11 had alleles that were unique to the risk haplotype. Each of these SNPs showed a significant level of TDT on its own (even after conservative correction for multiple testing) (Table 5). The 11 SNPs were essentially identical in their information content by virtue of being in nearly total linkage disequilibrium with one another (with the allele at one SNP determining the allele at all others on nearly every phased chromosome). Genotyping of the remaining 256 SNPs in the core region are ongoing and it is anticipated that approximately a dozen more SNPs unique to the risk haplotype with properties identical to these first 11 will be identified.

Further study to identify which SNP is responsible for increased susceptibility to CD is still required. The limits of genetic mapping have likely been reached for this European-derived population. It is possible that higher resolution mapping could be achieved with a follow-up study in individuals from an older population with shorter average blocks of LD, such as an African-derived population (Reich, D. E. et al., Nature (in press) (2001)).

Biological study may be required to discriminate among the SNPs unique to the risk haplotype. The SNPs genotyped to date include all those in or near known or predicted genes, with the SNPs remaining to be genotyped being in anonymous sequence. Unfortunately, there is no ‘smoking gun’ among these candidates. Of the 11 SNPs unique to the haplotype, none are in genes of known function and none have obviously important functional consequences. It is entirely possible that the causative SNP plays a regulatory role that is not readily evident from the sequence.

TABLE 5
Summary of SNPs with significant TDT results.

| | Approximate | | | | | | | |
|---|---|---|---|---|---|---|---|---|
| | Physical | SNP | Transmitted | Frequency | | | | |
| SNP marker name | position¹ | type | allele | of allele² | T:U | X2 | p-value | Comment |
| IGR2055a_1 | 435.0 | G/T | G | 0.357 | 87:39 | 18.29 | 0.000019 | |
| IGR2060a_1 | 437.5 | G/G | C | 0.351 | 81:34 | 19.21 | 0.000012 | |
| IGR2063b_1 | 439.0 | G/G | G | 0.359 | 87:37 | 20.16 | 0.000007 | Predicted to |
| | | | | | | | | cause a |
| | | | | | | | | missense |
| | | | | | | | | change in a |
| | | | | | | | | GENSCAN- |
| | | | | | | | | predicted gene |
| IGR207a_1 | 446.5 | A/G | A | 0.364 | 48:16 | 16.00 | 0.000063 | Located within |
| | | | | | | | | the “E3” EST |
| | | | | | | | | which may be a |
| | | | | | | | | splice variant |
| | | | | | | | | of OCTN2 |
| | | | | | | | | (ref 9) |
| IGR2096a_1 | 455.5 | A/C | A | 0.349 | 75:32 | 17.28 | 0.000032 | |
| IGR2198a_1 | 506.5 | G/G | G | 0.364 | 87:41 | 16.53 | 0.000048 | |
| IGR2230a_1 | 522.5 | C/T | T | 0.415 | 67:28 | 16.01 | 0.000063 | |
| IGR2277a_1 | 546.0 | A/G | G | 0.417 | 79:37 | 15.21 | 0.000096 | |
| IGR3O8la_1 | 609.0 | G/T | G | 0.338 | 79:35 | 16.98 | 0.000038 | |
| IGR3096a_1 | 616.5 | C/T | C | 0.429 | 89:42 | 16.86 | 0.00004 | Located within |
| | | | | | | | | EST cluster |
| | | | | | | | | HS.59096 |
| IGR3236a_1 | 686.5 | G/T | 1 | 0.383 | 79:39 | 13.56 | 0.00023 | |

¹Position (kb) on the 983 kb reference sequence.
²Frequency of allele calculated from the untransmitted parental chromosomes.
³The number of transmitted (1) chromosomes to untransmitted (U) chromosomes.

In summary, four statistically independent lines of evidence provide powerful support for this IBD5 susceptibility allele: (1) significant linkage in families with multiple affected siblings (set A, p<10⁻⁴), (2) significant association to CD trios as measured by TDT analysis of SSLP-based haplotype data (set B, p=0.00016), 3) significant association to CD trios as measured by TDT analysis of SNP based haplotype data in an independent set of trios (set C2, p=0.0002) (Combining these two association studies in the Toronto population yields p<2×10⁻⁷), and (4) significant replication of the findings in an independent population from Quebec (set D, p<0.05). The present study has resulted in the mapping of a risk allele for early-onset CD to a region of 250 kb containing about 10 genes and to a haplotype within this region that shows highly significant over-transmission from heterozygous individuals to affected offspring. LD mapping has thus succeeded in narrowing down a large linkage peak of 18 cM to a common haplotype spanning approximately 250 kb and in identifying a very short list of candidates, although identifying the specific variant/variants responsible for the CD phenotype could require biological experimentation.

### EXAMPLE 2

Determining High-Resolution Haplotype Structure in Chromosome 5q31

As described herein, a 500 kb region on human chromosome 5q31 is implicated as containing a genetic risk factor for Crohn disease (CD). High-density SNP discovery across the region was performed and 97 common SNPs in 129 trios from a European-derived population from Toronto, Canada were genotyped. The study focused primarily on those SNPs with minor allele frequency >5%. The results thus describe 258 chromosomes transmitted to CD patients and 258 normal, untransmitted chromosomes.

The genotype data provide a high resolution picture of the pattern of genetic variation across a large genomic region, with a marker density of 1 SNP every ˜5 kb. To obtain a clearer picture, the study of chromosome 5q31 focused on systematically identifying the underlying haplotypes. It became evident that the region could be largely decomposed into discrete haplotype blocks, each with striking lack of diversity (FIG. 2). Although the initial focus was on untransmitted control chromosomes; however, the same haplotype structure was seen in the chromosomes transmitted to CD patients, with the only difference being that one of the haplotypes was enriched in frequency reflecting its association to CD. Because the underlying ancestral structure is the same in both groups, combined data from all chromosomes (transmitted and untransmitted) is presented here. All of the underlying data for both groups are available at the website noted in the methods section which follows.

The haplotype blocks in this region span 10 to 100 kb and contain multiple (5 or more) common SNPs. The blocks have only a handful of haplotypes (2-4), which show no evidence of being derived from one another by recombination and which together account for nearly all chromosomes (>90% in all cases) in the sample. For example, an 84 kb block shows only two distinct haplotypes that together account for 97% of the observed chromosomes (Table 6). The lack of diversity is readily seen from the fact that the probability that a haplotype block is homozygous (for the SNPs genotyped) ranges from 30-70%.

TABLE 6

Haplotypes of SNPs 1118a_1 through 1286a_1

| | |
|---|---|
| Haplotype | Observations |

| | | | |
|---|---|---|---|
| G G A C A A C C | 283 | 83.2% | Haplotype A |
| A A T T C G G G | 40 | 11.8% | Haplotype B |
| G A T T A G C C | | | |
| G G T C A G C C | | | |

(another 13 chromosomes (2.5%) were observed which matched haplotypes A or B at all alleles except one and may represent genotyping error)

The discrete blocks are separated by intervals in which multiple independent historical recombination events appear to have occurred, giving rise to greater haplotype diversity for regions spanning the blocks. Such recombination events are denoted in FIG. 2 by lines connecting haplotypes. The recombination events appear to be clustered, with multiple obligate exchanges needing to have occurred between blocks but little or no exchange within blocks. For example, in the aforementioned 84 kb block (Table 6), not a single apparent recombinant between the two major haplotypes was observed (despite the fact that such a recombinant would be readily evident because the haplotypes differ at all SNPs examined). The clustering is suggestive of local hotspots of recombination (Templeton, A. R. et. al., Am. J Hum. Genet. 66:69-83 (2000); Jeffreys, A. J., et al., Hum. Mol. Genet. 9:725-733 (2000); Smith, R. A. et al., Blood 92:4415-4421 (1998). Although there is detectable recombination between blocks, it is modest enough that there is clear long-range correlation (that is, linkage disequilibrium) among blocks. The haplotypes at the various blocks can be readily assigned to one of four ancestral long-range haplotypes (A, B, C, D). Indeed, 38% of the chromosomes studied carried one of these four haplotypes across the entire length of the region.

A mathematical approach to formally define the block structure was developed by using a Hidden Markov Model (HMM). The HMM simultaneously assigns every position along each chromosome to an ancestral haplotype (in this case, A, B, C, or D) and estimates the maximum-likelihood values of the “historical recombination frequency”(⊖) between each pair of markers. The quantity ⊖ provides a convenient summary of the degree of haplotype exchange across inter-marker interval and relates directly to the conventional measures of LD such as D′ (methods). An alternative measure is the joint probability of homozygosity (Sved, J. A., Theor. Pop. Biol. 2:125-141 (1971)). In the case at hand, the discrete block structure is evident from the fact that ⊖ is estimated at <1% for 73 of the inter-marker intervals, 1-4% for the 14 of the intervals, and >4% for only 9 of the intervals.

It is important to consider whether the process of SNP selection could have influenced these results. The SNPs studies were ascertained by essentially complete resequencing of 8 individuals. Because the screen included 7 CD patients, it was considered whether this significantly biased the SNP ascertainment. To examine this, a 100 kb region of sequences in the center of the region was selected and the current method of SNP detection was compared to the SNPs identified by the International SNP Map Working Group (ISMWG: The International SNP Map Working Group, Nature 409: 928-33 (2001)). In the screened sequence, the ISMWG reported 54 SNPs and the present study identified 47 of them (86%). This is consistent with the independent observation that about 85% of SNPs identified by the ISMWG in its broad multiethnic panel were found to be polymorphic in a Caucasian panel and suggests that there is no significant bias in the ascertainment of SNPs used here due to ascertainment on primarily affected individuals. In addition, 150 SNPs in this region not reported by the ISMWG were discovered.

The analysis above included only SNPs with minor allele frequency >5%. 6 rarer SNPs that occurred with minor alleles occurring 10-20 times in the data set of >500 chromosomes were not studied. This rare allele fell exclusively or nearly exclusively on one of the major haplotype patterns. This indicates that the rare SNPs have a more recent ancestry and that they contribute to diversity only by creating rare subtypes of common haplotype patterns. Since each individual chromosome has not been sequenced completely, it is likely that additional rare variants are carried on some of the chromosomes sampled. Thus, when limited haplotype diversity is described, complete sequence identity is not implyed, but rather that chromosomes fall into a small number of deep clades. Chromosomes within a lade may differ at one or a few rare SNPs, while chromosomes in different clades differ at many SNPs.

It was noted that SNPs at CpG sites were initially eliminated from the analyses because the higher mutation rate at such sites (Krawczak, M., et al., Am. J. Hum. Genet. 63:474-88 (1998); Nachman, M. W. et al., Genetics 156:297-304 (2000)) might cause recurrent mutations and confound the analysis. Reviewing the 16 high frequency CpG SNPs genotyped in this region, it is noteworthy that 13 have alleles that align perfectly with the haplotype patterns described in FIG. 1. Only 1 of the 16 would add significantly to the overall heterozygosity of the block in which it fell.

The analysis of this region of chromosome 5q31 in a European-derived population indicates that: the region may be largely parsed into discrete blocks of 10-100 kb; that each block has only a few common haplotypes; and that the haplotype correlation between blocks gives rise to long-range linkage disequilibrium.

Additional Methods

The individual studies and genotyping methodologies are as described for Example 1. To ensure ability to reconstruct multimarker haplotypes, SNPs for haplotype analysis were selected from the set of markers for which full genotypes were available for all members of 85 or more trios. To eliminate markers likely to contain significant numbers of undetected genotyping errors, markers not in Hardy-Weinberg equilibrium (p<0.5) or those for which more than 10 Mendelian inheritance errors were detected were excluded from this analysis. SNPs at CpG sites were not included in the initial analysis to prevent potential confounding of common haplotype patterns from recurrent mutation. Additionally, rare SNPs (minor allele frequence <5%) were not included in the initial analysis.

Haplotype percentages in FIG. 2 were computed by using haplotypes generated by the TDT implementation in GENEHUNTER 2.0 (Daly, M. J., et. al., Am. J. Hum. Genet. 63:A286 (1998)) followed by use of an EM-type algorithm (Dempster, A. P., et al., J. R. Stat. Soc. 39:1-38 (1977); Excoffier L., et al., Evol. 12:921-927 (1995)) to include the minority of chromosomes that had one or more markers with ambiguous phase (i.e., where both parents and offspring were heterozygous) or where one marker was missing genotype data. Use of Clark's method (Clark, A. G., Mol Biol Evol 7:111-122 (1990)) or simply counting only fully informative, phase-known, haplotypes provided essentially identical answers since within each block the vast majority of chromosomes were fully reconstructed without ambiguity from the parental data.

Regions of low haplotype diversity were initially identified in the following fashion: five-marker haplotypes for all consecutive sets of 5 markers were generated; the observed haplotypic heterozygosity (HETobs) and expected haplotypic heterozygosity (HETexp) (given allele frequency and assuming equilibrium) were tallied; and each 5 marker window was assigned a score S5=HETobs/HETexp. A smaller value therefore represents lower diversity of haplotypes compared with expectation. Windows with locally minimal scores were then expanded or contracted by adding or subtracting markers to the ends to find the longest local minimum window. Boundaries between these windows (which we call “blocks”) were examined, the most common connections between haplotypes considered to be the “ancestral haplotype class” (displayed on the same line in the same color in FIG. 2), and cases in which a high frequency (>2%) haplotype is observed which represents a connection between two different “ancestral classes” are shown by a line connecting those classes across that interval.

The observation that over long distances, most haplotypes can be described as either belonging to one of a small number of common haplotype categories or as a simple mosaic of those categories suggested the use of an HMM in which haplotype categories were defined as states. Observed chromosomes were assigned to those hidden states (allowing for missing/erroneous genotype data) and the transition probability in each map interval was simultaneously estimated using an EM algorithm and making the simplifying assumption that there was one transition probability for each map interval (the aforementioned probability of historical recombination ⊖) rather than allowing specific transition probabilities from each state to each state. The output of this method was a maximum-likelihood assignment to haplotype category at each position (which can be used to compute multi-allelic D′, TDT, etc.) And maximum-likelihood estimates of ⊖ indicating how significantly recombination has acted to increase haplotype diversity in each map interval. The use of probabilities of recombination in this context (Sved, J. A., Theor Pop Biol 2:125-141 (1971)) has a simple relationship with the most commonly used measure of gametic disequilibrium (D′). If we consider two SNPs at a time before any recombination (or other type of event) has occurred to create a fourth haplotype as follows:

```
                                                SNP 2
                                  Allele 1 Allele2
SNP1 Allele 1 a b
      Allele 2 c=0 d
```

It is apparent that D′ (=(ab−bc)/((a+c)(c+d)) for this table configuration) is equal to 1 (full disequilibrium). Many generations later, all recombination that has occurred between the two markers can be collapsed into a single value—the probability that a modern chromosome has undergone recombination at any time between those two markers. Let 1−⊖ represent the probability that no recombination has taken place at any time between these two markers. At this time, the table of haplotype frequencies will have changed to:

```
                                                SNP 2
                                  Allele 1 Allele2
SNP1 Allele 1 a-ad⊖ b+ad⊖
      Allele 2 ad⊖ d-ad⊖
```

Add now D′ =(a−d⊖)(d−d⊖_−bd⊖−(d⊖)²/ad which reduces to (ad−ad⊖)/ad and thus D′=(1−⊖). ⊖ here (⊖ᵣₑₐₗ) differs from the observed rates reported in Table 2 (⊖_{obs}) since some recombinations occur between chromosomes with identical local haplotypes—however the observed values are trivially corrected by the local homozygosity to produce

### EXAMPLE 3

Analysis of Fifty-Four Genomic Regions in Four Population Samples

54 autosomal regions were studied, each with an average size of 250,000 bp, spanning in total 13.1 Mb of human genome sequence. To ensure that the results were represEntative of the autosomes, regions were selected at random, subject only to the availability of contiguous genomic sequence and an average density (in a core region of 150 kb) of one candidate SNP discovered by The SNP Consortium (TSC) project every 2 kb (a density approximating that of the genome as a whole (Sachidanandam et al., Nature 409:928-33. (2001)). A total of 5,884 candidate TSC SNPs were selected for study (Only SNPs from the TSC discovery project were selected, as these were discovered using a uniform protocol in a multiethnic sample of known composition. SNPs were spaced no closer than 500 bp apart (designed to exclude multiple SNPs sampled from the same sequencing read)). Other than these criteria, all TSC SNPs were accepted—no other filtering was applied.

The SNPs were genotyped in four population samples: (1) a European-derived sample of 93 individuals from the (Utah) CEPH resource (four grandparents, two parents and one or two offspring from each of 12 multigenerational pedigrees); (2) an Asian sample of 42 unrelated individuals (10 Chinese, 32 Japanese); (3) an African sample of 90 individuals comprising 30 mother-father-offspring trios from the Yoruba in Nigeria; and (4) an African-American sample consisting of 50 unrelated individuals (CEPH samples were from the Utah pedigrees; specific sample identifiers are available on The SNP Consortium website. The Asian and African-American samples were obtained from the Coriell Cell repository, with 10 Chinese and 10 Japanese drawn from the Human Variation Panel, and an additional 22 Japanese control samples from the American Diabetes Association GENNID study. The African-American samples constituted the HD50AA diversity panel. The Yoruban samples are healthy individuals from a population-based study in Nigeria. Multiplex PCR was performed in five microliter volumes containing 0.1 units of Taq polymerase (Amplitaq Gold, Applied Biosystems), 5 ng genomic DNA, 2.5 pmol of each PCR primer, and 2.5 μmol of dNTP. Thermocycling was at 95 C. for 15 minutes followed by 45 cycles of 95 C. for 20 s, 56 C. for 30s, 72 C. for 30 s. Unincorporated dNTPs were deactivated using 0.3U of Shrimp Alkaline Phosphatase (Roche) followed by primer extension using 5.4 pmol of each primer extension probe, 50 μmole of the appropriate dNTP/ddNTP combination, and 0.5 units of Thermosequenase (Amersham Pharmacia). Reactions were cycled at 94 C. for 2 minutes, followed by 40 cycles of 94 degrees for 5 s, 50 degrees for 5 s, 72 degrees for 5 s. Following addition of a cation exchange resin to remove residual salt from the reactions, 7 nanoliters of the purified primer extension reaction was loaded onto a matrix pad (3-hydroxypicoloinic acid) of a SpectroCHIP (Sequenom, San Diego, Calif.). SpectroCHIPs were analyzed using a Bruker Biflex III MALDI-TOF mass spectrometer (SpectroREADER, Sequenom, San Diego, Calif.) and spectra processed using SpectroTYPER (Sequenom). These four population samples were chosen to explore a wide range of human diversity, but they should not be regarded as a comprehensive sample of global or continental diversity. Pedigrees were used (in the analysis of European and Yoruban samples) because they provide direct observation both of haplotype phase (the arrangement of alleles on a single physical chromosome) and of genotyping errors (based on violations of Mendelian inheritance).

Genotyping was performed by primer extension of multiplex products and detection by MALDI-TOF mass spectroscopy (Tang, K., et al., Proc Natl Acad Sci U S A 96:10016-10020 (1999). Multiplex genotyping assays were successfully designed for 87% of all SNPs (Primers and probes were designed in multiplex format (average 3.4 fold multiplexing) using SpectroDESIGNER software (Sequenom, San Diego, Calif.) All primer and probe sequences are available at the TSC website), with the remaining 13% rejected by the automated algorithm because of high repeat content adjacent to the SNP base. Of 5,157 SNPs for which probes were designed, genotyping was successful for 4,410 (85%) (Successful genotyping assays were defined as those in which ≧75% of all genotyping calls were obtained and all quality checks passed (see below). Although 75% was a lower threshold, on average we obtained 94% genotypes attempted for each SNP. Although 85% of assays were successful in at least one population, success of assays in any one population ranges from 72% to 80%. Assays that provided fewer than 75% of genotypes were repeated once in the laboratory and consensus genotypes calculated; if not converted into successful assays, a single round of primer redesign and repeat testing was performed.). This provides an average density of one candidate SNP successfully genotyped every 3 kb across these regions.

The accuracy of the genome assembly and of the individual genotypes are critical for the study of multimarker haplotypes. Incorrect map locations and as little as 1-2% genotyping error can obscure or confuse the underlying haplotype patterns. To eliminate regions with questionable genome assemblies, we compared the relative map positions of each SNP across multiple genome builds and independent assemblies (NCBI and UCSC); one region was withheld from analysis due to inconsistencies of relative map positions. Recently duplicated (paralogous) genomic regions based on a high proportion of candidate SNPs demonstrating uniformly heterozygous “genotypes” were eliminated: two of the 53 regions were withheld from analysis on this basis. In the remaining 51 regions, accuracy of genotype calls was ≈99.7% as assessed by Mendelian inheritance and by genotypes performed in duplicate.

Hardy-Weinberg equilibrium was evaluated for each population sample, and markers were rejected if they violated H-W equilibrium (a conservative threshold of p<0.01 was set, uncorrected for multiple comparison). In two clusters, a high proportion (>50%) of markers had high heterozygosity, presumably reflecting duplicated genomic loci; these clusters were withheld from further analysis. Of the remaining 51 clusters, only 1.8% of markers were rejected for violations of HW equilibrium. Two independent tests were used to estimate error rates, providing identical conclusions. First, we observed 1,068 Mendel errors in 598,466 polymorphic genotypes examined in pedigrees, providing a raw error rate of 0.18%. This provides only a lower bound on the error rate, however, since only a subset of genotyping errors (25-50%) result in Mendel errors. 970 SNPs were also genotyped more than once in the same DNA samples, and 1,375 discrepancies were found in 394,688 genotypes performed. This measure of reproducibility provides an independent error rate estimate of 0.4%.

The SNP Consortium discovered SNPs as random heterozygous positions (Altshuler, D., et al., Nature 407:513-516 (2000); Mullikin, J. C., et al., Nature 407:516-520 (2000)) in a multiethnic collection of DNA samples (Collins, F. S., et al., Genome Res 8:1229-1231 (1998)), providing a collection of SNPs that are diverse with regard to allele frequency and population distribution. Of SNPs successfully genotyped, 89% were polymorphic in at least one of the four population samples. This high rate of polymorphism in independent samples directly demonstrates that the vast majority of positions that are heterozygous between two randomly chosen genomes are attributable to common (>1% minor allele frequency) variants. These data also confirm that ≈90% of TSC SNPs can be validated when assayed in a sample of appropriate sizeand diversity.

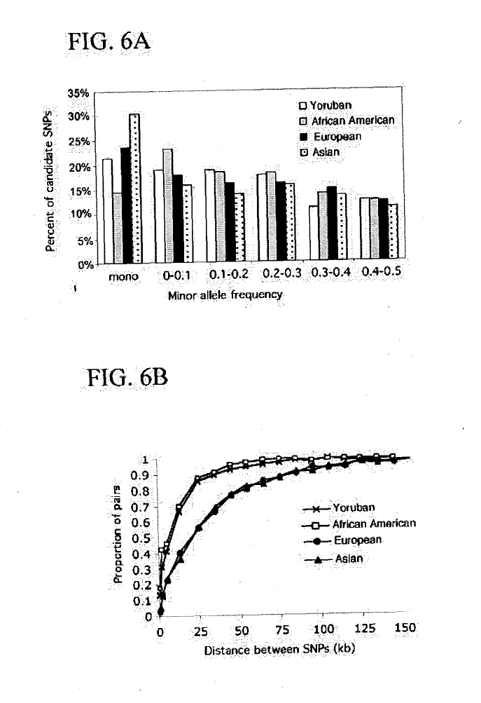

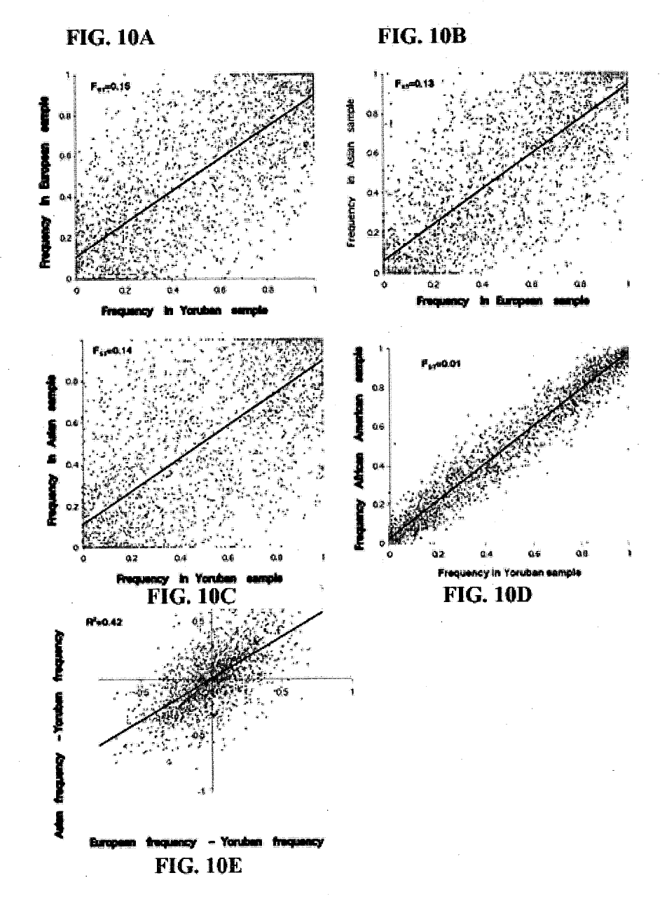

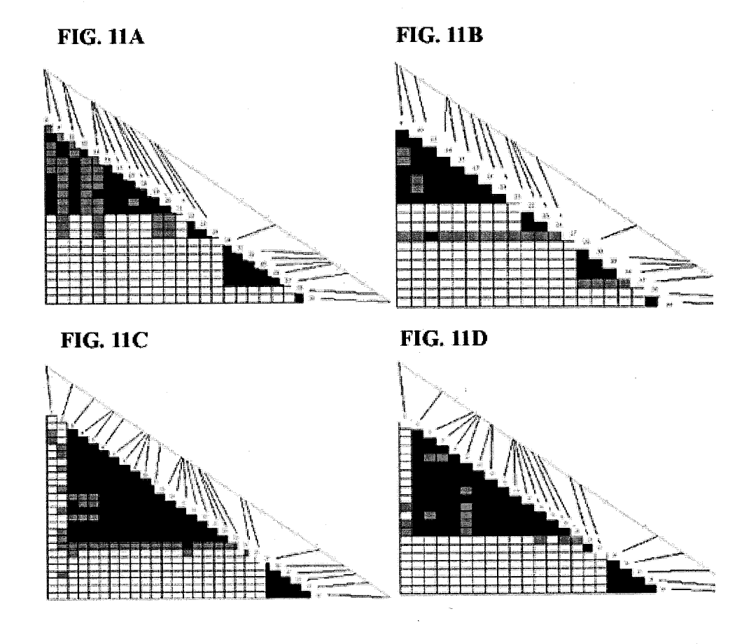

The fraction of SNPs that were polymorphic was found to vary substantially across population samples (normalized for sample size by randomly selecting 64 chromosomes from each sample): from 68% in the Asian sample to 86% in the African-American sample (FIG. 6A). Interestingly, the difference in frequency distribution among populations is entirely attributable to lower frequency alleles, as the proportion of SNPs with high frequency (minor allele frequency ≧0.2) was very similar in all groups (41-48%). In addition, although most variants (59%) were observed in all four populations, there are dramatic differences in the allele frequencies of individual SNPs across samples (FIG. 10B). The level of allele frequency scatter can be summarized by the metric FST (FIGS. 10A-10E), which ranged from 0.006 (for the comparison of the Yoruban and African-American samples) to 0.20 (Asian and Yoruban samples), consistent with prior estimates of population differentiation (Cavalli-Sforza, L. L., et al., The history and geography of human genes (Princeton University Press, Princeton, N.J., 1994)). The difference in allele frequencies between the European and Yoruban samples was highly correlated with the difference in allele frequencies between the Asian and Yoruban samples (FIG. 10C) supporting a common origin of the European and Asian population samples (Cavalli-Sforza, L. L., et al., The history and geography of human genes (Princeton University Press, Princeton, N.J., 1994)).

### EXAMPLE 4

Distribution of Linkage Disequilibrium Among Pairs of Markers

Patterns of linkage disequilibrium and haplotypes across each region were studied. The analysis is first outlined using one population sample (the European sample), and then the results are described across the four population samples. The analysis consisted of two steps: defining the patterns of historical recombination across each region; and, for segments inherited without significant historical recombination, examining the diversity of common haplotypes.

Linkage disequilibrium was measured using the normalized measure D′ (Lewontin, R. C., Genetics 49:49-67 (1964)), which reflects the history of recombination in the ancestry of a pair of polymorphic markers (Daly, M. J., et al., Nat Genet 29:229-232 (2001)). High values of D′ indicates pairs of SNP alleles that have been inherited without recombination since their shared ancestor; conversely, low values of D′ indicate substantial recombination. Other measures, such as r², can also be used to describe the correlation in state of randomly chosen allele pairs. Such measures are directly proportional to the power of association studies performed using markers chosen at random with regard to the underlying haplotype structure; distributions of R² are presented as FIG. 10. By first mapping out sites of historical recombination and the specific haplotypes observed, however, it is possible to select markers that carry substantially more information about the underlying sequence variation. As this approach results in more efficient association studies (as compared to the random selection of markers),r values underestimate the maximal power of empirically guided, haplotype-based association studies. Because there is significant sampling variation in the observed values of D′, 95% confidence intervals of D′ were calculated for each pair of markers with minor allele frequency ≧0.2. (Confidence limits were determined by calculating a probability distribution for D′, given the observed two marker gamete counts. The upper and lower limits represent the tails of that distribution.) “Strong LD” was defined as that for which the 95% confidence interval for the D′ value was consistent with complete LD (defined as D′ ≧0.98) and excluded a value below 0.7. Conversely, “strong evidence of historical recombination” was defined as D′ values for which the confidence interval excludesd values of D′ of 0.9 and higher. (D′ values slightly below 1.0 can be due to many processes other than recombination.) At very short distances, for example, deviations from complete LD are likely attributable to gene conversion rather than recombination (Frisse, L. et al., Am J Hum Genet 69:831-43. (2001); Ardlie, K. et al., Am J Hum Genet 69:582-9 (2001)), although recurrent mutation and genotyping error also contribute to this value. For example, with 96 chromosomes sampled and a minor allele frequency of 0.20, a single genotyping error or rare gene conversion event reduces D′ from 1.0 to 0.8.) In this framework, some marker pairs (primarily those with lower frequency) provide inconclusive information, because the confidence interval on D′ values is too wide to draw a conclusion—that is, the available data are not adequate to definitively assess historical recombination between the SNPs. Given the large number of chromosomes examined in this study, however, only a small fraction of high frequency marker pairs (varying between 3 and 25% at the different distances examined) failed to provide conclusive information about historical recombination.

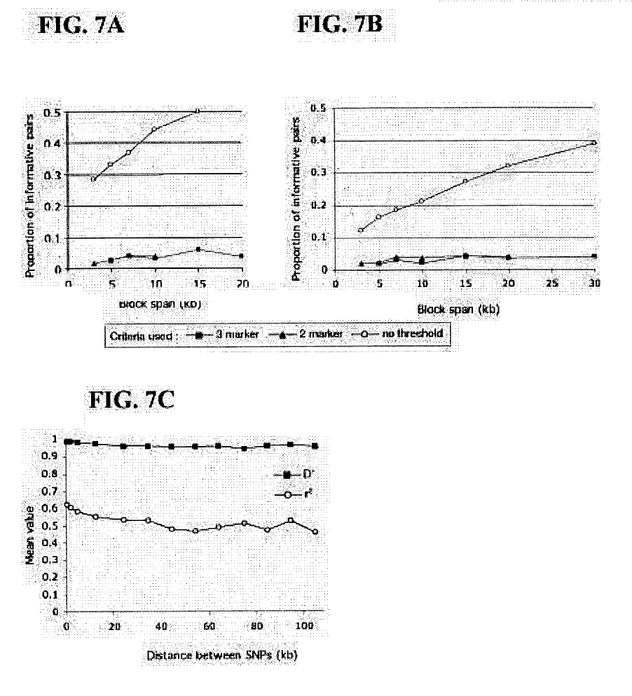

FIGS. 7A-7C presents the distribution of D′ for high-frequency SNPs (minor allele frequency ≧0.2) separated by distances of 500 bp-200,000 bp in the European sample. Consistent with previous reports, tremendous scatter was observed in the magnitude of D′ for pairs separated by any given distance (FIG. 7A). When the confidence interval is considered on the estimate of D′ (as described above), however, the pattern becomes substantially clearer. For closely linked markers (those separated by 500 bp-1,000 bp), 96% of informative pairs demonstrate strong LD, reflecting the small amount of recombination in the history of the alleles (FIG. 7B). Conversely, at very short distances, only 4% of informative marker pairs show strong evidence of historical recombination (FIG. 7B). Furthermore, a significant fraction of marker pairs demonstrate strong LD even when separated by substantial distances (FIG. 7B): 45% of pairs show strong LD when separated by 25 kb, and 6% of pairs over distances of 100 kb. These “strong” LD values are all statistically significant: in this sample, not a single informative, unlinked marker pair (of 1,378 examined) showed strong LD (as defined above).

### EXAMPLE 5

Fine Structure of Haplotype Blocks

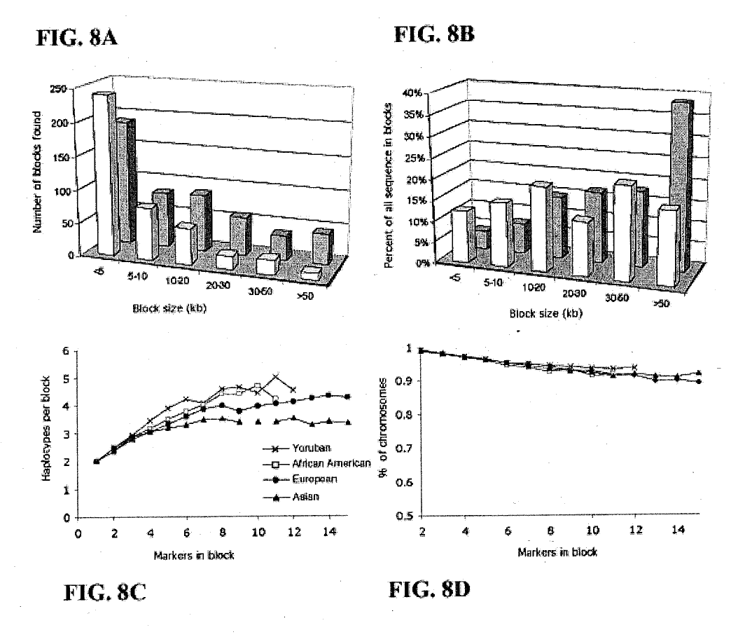

To characterize the fine structure of haplotypes responsible for the overall distribution of D′ values, the local correlation among D′ values for each region in the European sample was examined. Typical examples of regions are shown in FIGS. 8A-D with the entire data set presented as FIGS. 11A-D. Visual inspection of the data revealed many clusters of adjacent markers over which all pairs demonstrated strong LD, bounded by intervals over which D′ drops abruptly. Where many adjacent markers show strong or complete LD (measured using D′), historical recombination has been rare or absent across the region during the history of the sample. This observation suggested a simple and objective definition of a “haplotype block”: a region over which historical recombination (as measured by the distribution of D′ values) mirrors that typically observed for marker pairs separated by very short distances (<1,000 bp), and does not substantially decline as a function of the distance separating marker pairs. The data was systematically examined for sets of contiguous markers that satisfied these criteria. This involved two types of analyses, depending on the density of markers obtained across each region. First, the data was directly examined for runs of consecutive markers for which the desired proportion of informative pairs showed strong LD.

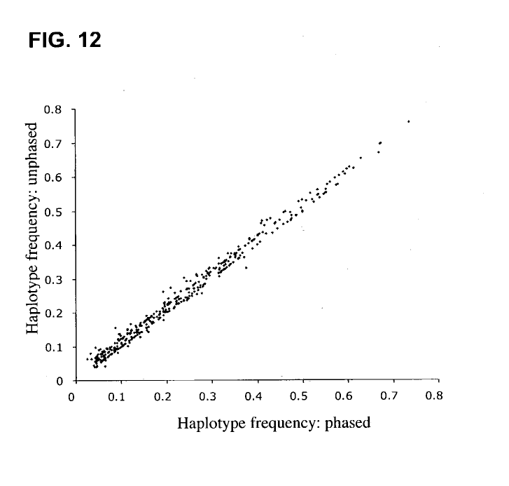

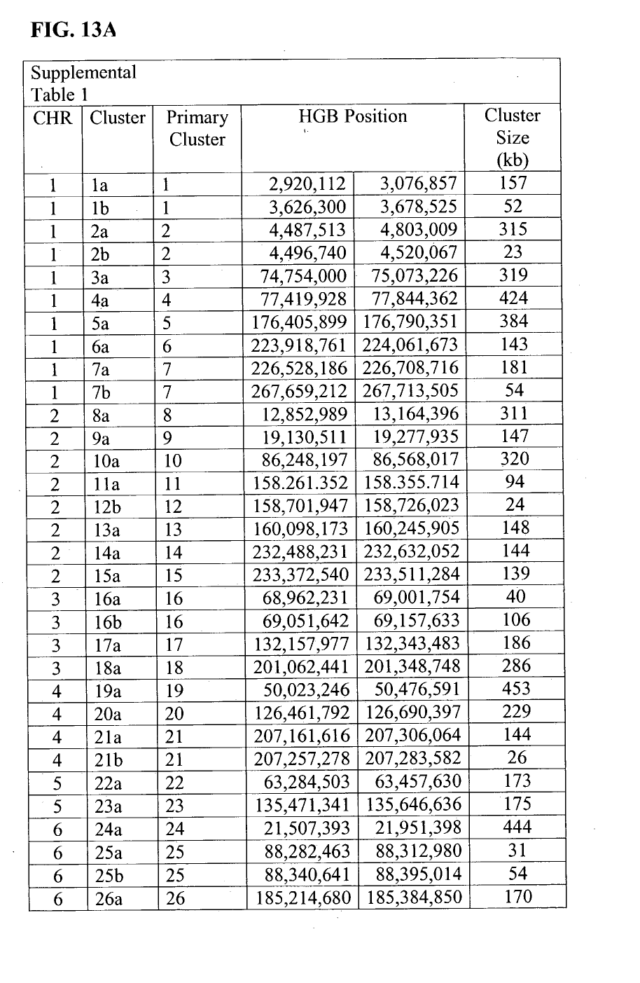

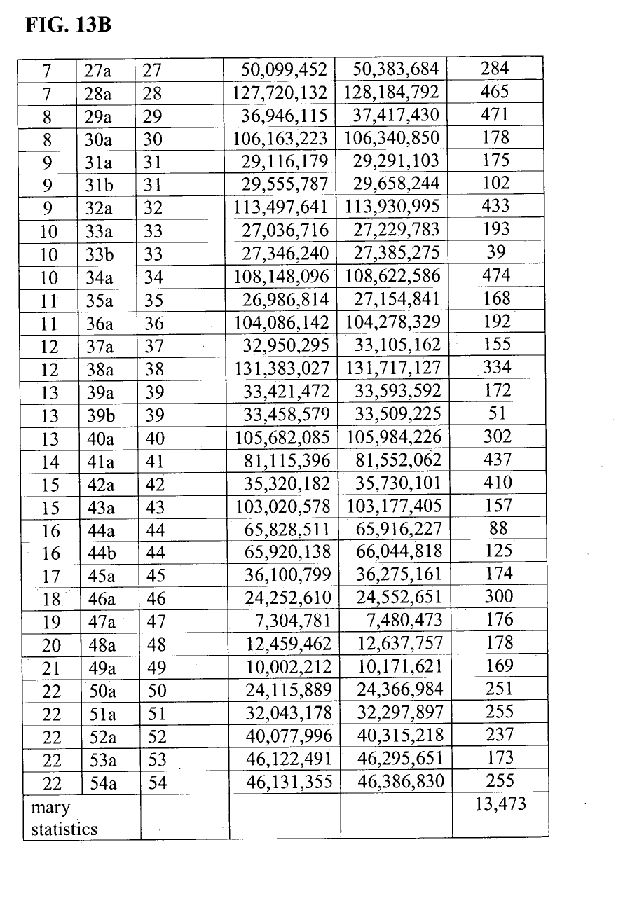

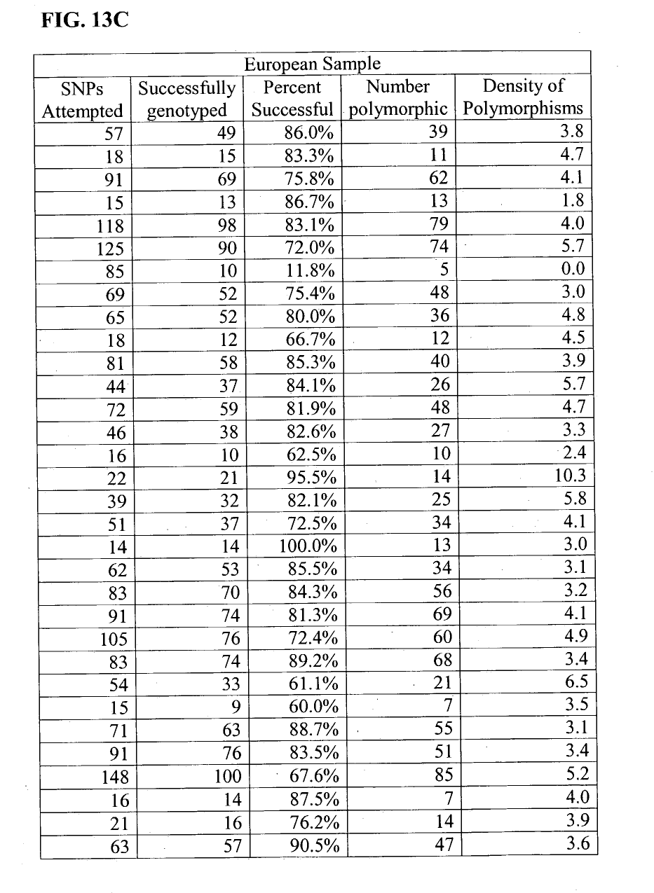

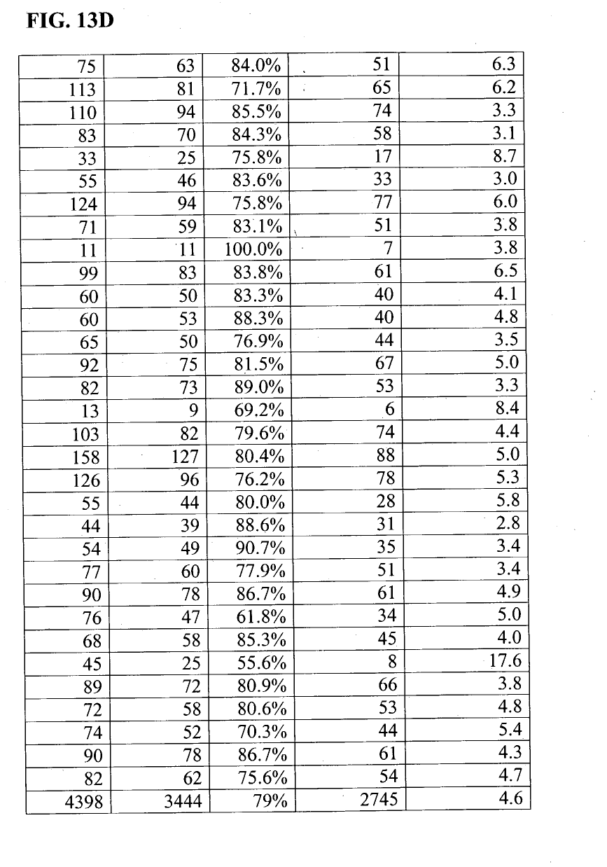

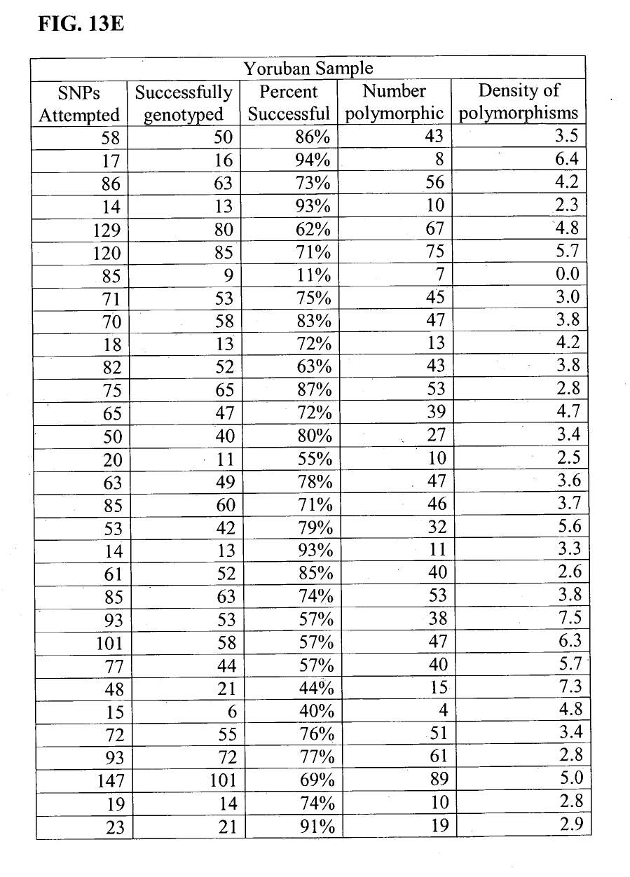

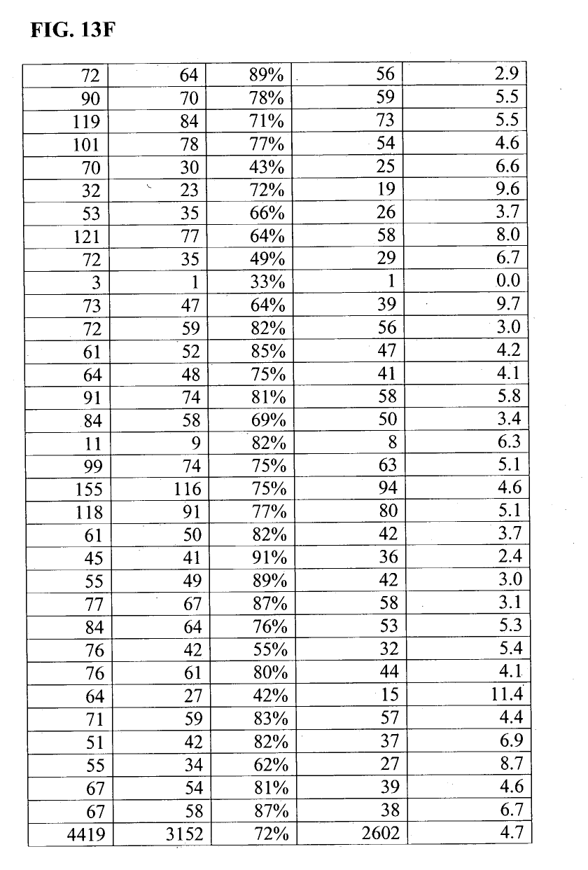

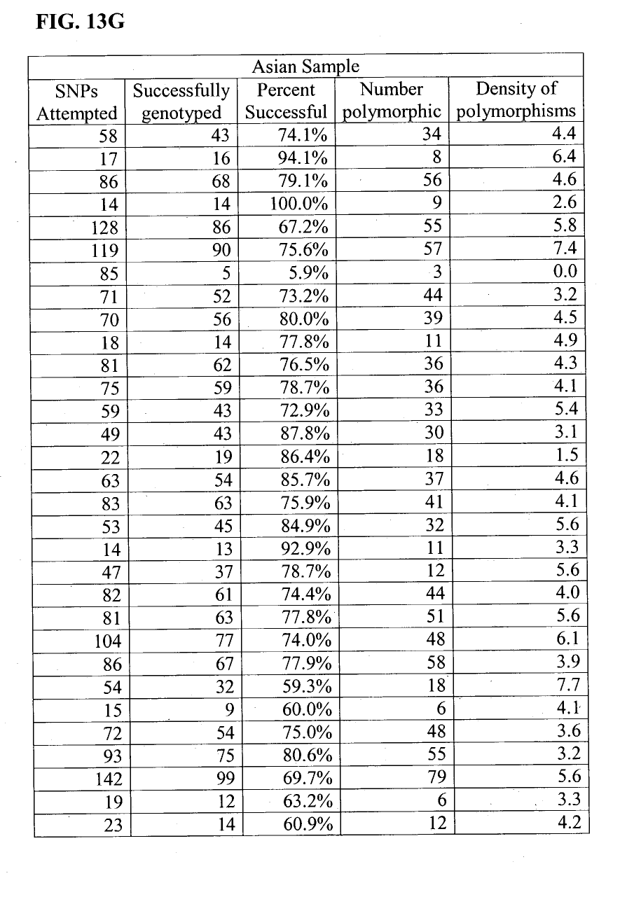

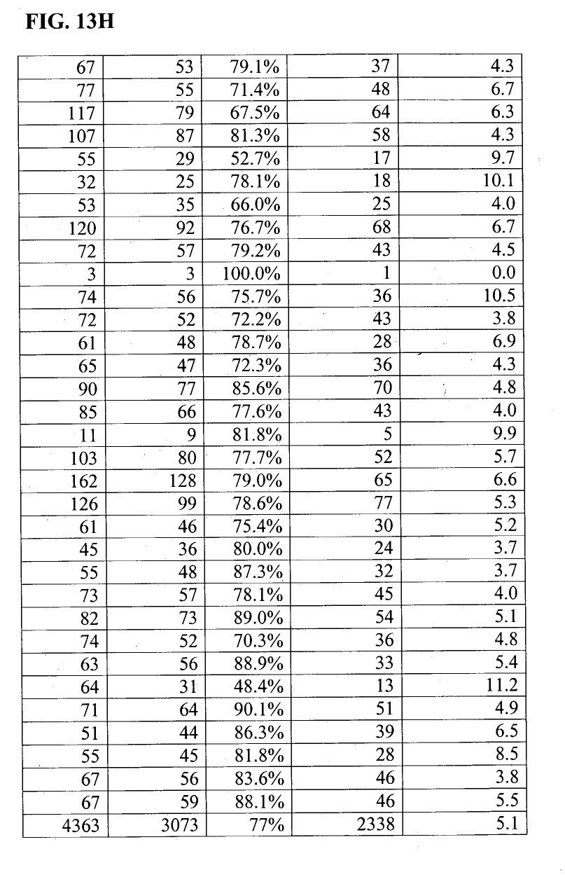

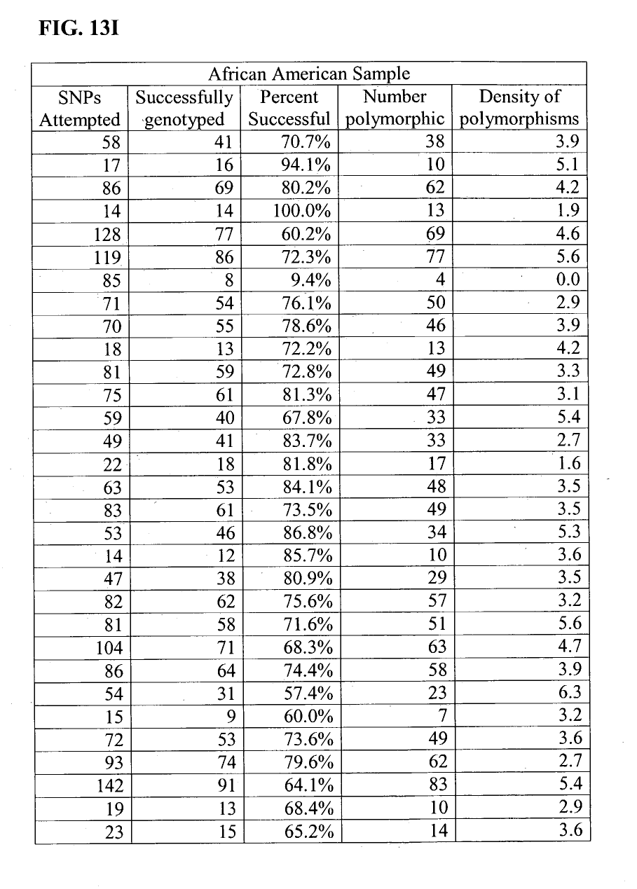

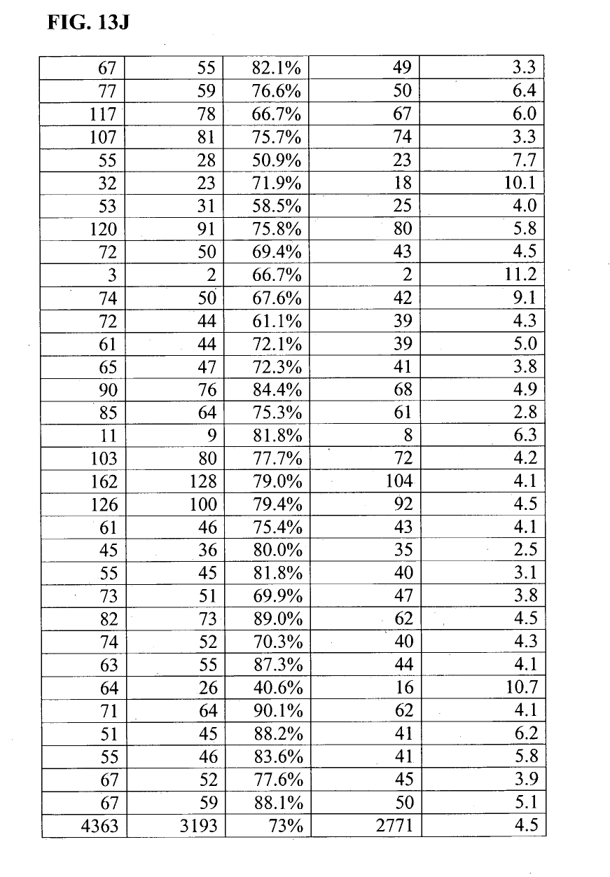

Second, to examine regions where marker coverage was more sparse (and thus the simple test not possible), the entire data set was queried to determine whether a framework of markers could suffice to identify regions within which the required proportion of internal pairs showed strong LD. That is, a subset of markers was sampled to characterize each region (based on D′ values, confidence limits, and marker spacing), and then queried the withheld internal markers (not used to characterize the region) for their distribution of D′ values. Based on the distribution of D′ values for these internal markers, it was possible to identify criteria for framework markers sufficient to identify blocks that (in aggregate) meet the proposed criteria (FIG. 12). (The final block definition was selected to obtain ≦7% of internal pairs showing strong evidence of recombination, which is the fraction of pairs showing recombination for pairs separated by ≦1,000 bp (weighted across the four population samples). Specifically, ≧93% of informative marker pairs across the region must show strong LD, and ≧50% of all markers in the block informative. For more closely-spaced markers, even looser definitions could suffice (FIG. 12): at separations <5 kb, a pair of markers with a lower CL(D′)>0.50 and upper CL(D′)>0.98; at separations <20 kb, a triplet of markers having lower CL(D′)>0.5 and upper CL(D′)>0.98.) Using these definitions, the proportion of genome sequence that was contained within haplotype blocks was examined. The data for sets of evenly-spaced markers was sampled, selecting marker sets with separations ranging from 1,000 to 50,000 bp (For marker spacing x, each genomic region was divided into equally-sized bins with span of x, and examined for bins containing at least one marker with frequency >0.2. Each such set of evenly spaced markers was tested for the block definition described above.) At the highest density of markers examined (one highly polymorphic marker with minor allele frequency ≧0.2 every 1,000 bp), the vast majority of sequence examined (190 of 212 kb, or 90%) was found in blocks. As the spacing between adjacent markers is progressively increased, only larger blocks can be captured; thus, this procedure provides a measurement of the distributions of block sizes. Using a marker spacing of 5 kb, 73% of sequence (1.5 Mb of 2.1 Mb) was identified in blocks; at 20 kb, 41% (2.8 Mb of 6.8 Mb) was in blocks, and at 50 kb, 19% (1.8 Mb of 9.6 Mb) was in blocks. (Table 7)

TABLE 7
Model versus observed distribution of block sizes

| | African sample | | Non-African sample | |
|---|---|---|---|---|
| | Observed % | predicted % | observed % | predicted % |
| | of spanned | of spanned | of spanned | of spanned |
| block size | sequence | sequence | sequence | sequence |
| 0.5 | 12.4 | 6.3 | 4.4 | 1.8 |
| 5-10 | 15.3 | 15.1 | 7.4 | 5.2 |
| 10-20 | 20 | 31.5 | 14.9 | 15.2 |
| 20-30 | 12.8 | 21.8 | 16.6 | 16.6 |
| 30-50 | 22.2 | 19.1 | 18 | 26.9 |
| >50 | 17.4 | 6.3 | 38.7 | 34.2 |

The mean span of the markers contained within a block was 21 kb, with a range of 1-152 kb. Of course, the true size of each block must be larger than the span of markers used to identify it, because randomly selected SNPs will seldom fall exactly at the edge of a block. To estimate the true underlying distribution of block sizes, we performed two independent analyses. First, we estimated block sizes under the assumption that each block boundary exists at the mid-position of the gap between the last marker identified in the block and the first marker outside the block (with a maximum possible addition of 5 kb). Using this approximation, the average block size in the European sample is estimated to be 25 kb. Second, the data in Table 1 was fitted to a model in which the boundaries between blocks are 2 kb in width (Jeffreys, A. J., et al, Nat Genet 29:217-222 (2001)) and Poisson-distributed with a mean separation of d. The model provides a surprisingly good fit to the data in the European sample when d=27 kb (Table 8) The agreement between these two estimates (25 kb and 27 kb) suggests that they provide reasonably robust estimates of the underlying distribution of block sizes. The model predicts that in the European population more than 90% of the human genome sequence exists in blocks between sites of historical recombination, with 50% of the genome in blocks of 45 kb or larger, and more than 88% of the genome in blocks of 10 kb or larger.

TABLE 8

| | European sample | | Yoruban sample | |
|---|---|---|---|---|
| Spacing of | Observed % | predicted % | observed % | predicted % |
| informative | of spanned | of spanned | of spanned | of spanned |
| pairs | sequence | sequence | sequence | sequence |
| 1 | 89.6 | 89.7 | 74.9 | 82 |
| 5 | 72.6 | 83.5 | 62.1 | 72 |
| 10 | 65.5 | 64.5 | 45.9 | 45 |
| 30 | 32.3 | 31.7 | 12.5 | 11.6 |
| 50 | 18.7 | 15.7 | 3.8 | 3 |

### EXAMPLE 6

Haplotype Diversity Within Blocks

Having identified blocks with limited historical recombination the diversity of haplotypes in each block was examined In the absence of historical recombination (and barring genotyping errors), differences among haplotypes are due only to new mutations or gene conversion events since the common ancestor. Multi-marker haplotypes for each of the 250 blocks identified in the European survey, using all markers with minor allele frequencies ≧5% were constructed. (While marker frequencies of ≧20% were used to define blocks (since rare markers are often uninformative about historical recombination given the sample size employed), lower frequency markers can be employed for haplotype determination without bias.). The haplotype diversity of each block was quite limited, with an average of 4.2 common haplotypes (defined as ≧5% frequency) per block (FIGS. 8C and 8D) Exact matches to one of these few common haplotypes typically explained the vast majority (≈90%) of all chromosomes in the sample (FIGS. 8B and 8C). Since not all SNPs within each block were ascertained, it is possible that haplotype diversity was systematically underestimated. To address this possibility, the mean number of common haplotypes in each block as a function of the number of markers tested was noted. The mean number of common haplotypes quickly reaches an asymptote where six to eight SNPs have been typed in the block, and does not increase appreciably where as many as eighteen markers are used to define the block (FIG. 8C). The mean proportion of all haplotypes explained by perfect matches to one of these few common (≧5% frequency) haplotypes remains above 90% for blocks defined with as many as 18 markers (FIG. 8C) These data indicate that within regions characterized by limited historical recombination, a modest number of ancestral haplotypes explains 90% or more of all common sequence diversity in the European sample, and that these common haplotypes can be well-defined with as few as six to eight common SNPs per block (Since the number of markers in a block is correlated with the size of the block, it is possible that the plateau reflects an inverse relationship between block size and haplotype diversity; direct examination of block size and haplotype number reveals this is not the case (data not shown).

The exchanges between ancestral segments (historical recombination events) that are reflected in the current population were also examined. A block-like pattern of linkage disequilibrium could be attributable to the random genealogical process, with boundaries between blocks representing sites at which one or two historical recombination events happened to occur early in the history of the sample (and which are thus common in the current population, (Subrahmanyan, L. et al., Am J Hum Genet 69:381-95 (2001)). Alternatively, boundaries between blocks could be created by hotspots of meiotic recombination (Chakravarti, A., et al., Am J Hum Genet 36:1239-58 (1984); Jeffreys, A. J., et al, Nat Genet 29:217-222 (2001)). In the latter case (but not the former), many independent recombinant forms would often be represented across each block boundary.

The excess number of haplotype combinations (R_{H}) seen in adjacent blocks as compared to blocks with no recombination was computed. For example, if two adjacent blocks each contain three common haplotypes, but when considered together displayed 7 of the 9 possible combinations of those haplotypes, R_{H} would be 4 (the minimum number of recombination events required to create the observed haplotype combinations). FIG. 8D shows the distribution of R_{H} as a function of the distance spanned by adjacent block boundaries: the mean value of R_{H} is 3.1 for block boundaries spanning <3 kb, and then rises with increasing distances between the blocks. This indicates that where our interblock intervals are large (>5 kb) due to gaps in SNP coverage, more than one site of historical recombination has sometimes been spanned. However, even at the shortest inter-block interval (<3 kb), there are typically many independent recombination events observed. The large number of recombinant haplotypes often observed across block boundaries supports the idea that hotspots of recombination may play an important role in shaping human genome sequence variation. In summary, haplotype blocks—defined as regions that have undergone as little historical recombination as is typical for a 1 kb region—average 26 kb in size in the European sample, cover 90% or more of the human genome sequence, and typically contain only four or five common haplotypes that capture ≈90% of all chromosomes in the sample.

### EXAMPLE 7

Comparison of Haplotype Block Structure Across Populations

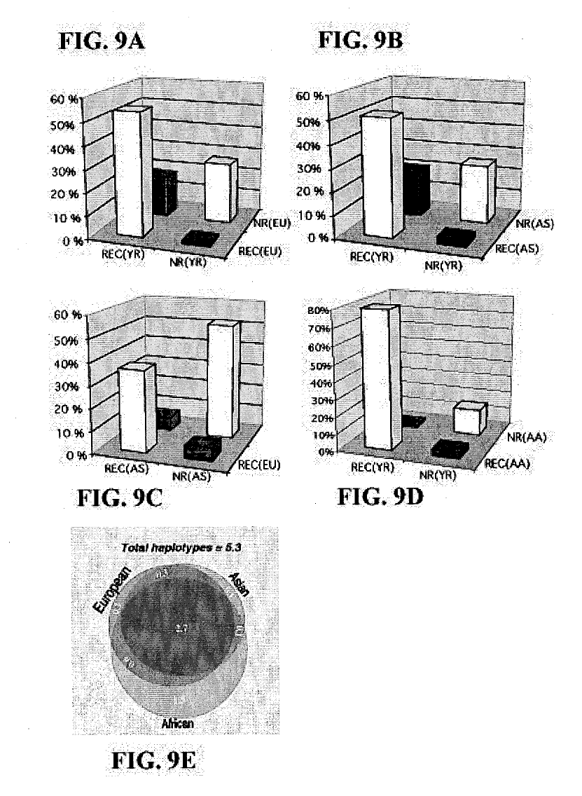

Using the framework developed above, the haplotype patterns among the four population samples were examined. The distribution of D′ values, using the same (confidence interval based) definitions of strong LD and strong evidence for historical recombination was studied. In each population sample, a similar and low proportion (4-12%) of informative marker pairs separated by less than 1,000 bp show strong evidence for historical recombination, although the values are higher for the Yoruban and African-American samples than for the European and Asian samples (FIG. 9A) In the Asian sample, furthermore, the proportion of pairs showing evidence of historical recombination increases with distance in parallel to that observed in the European sample. In contrast, the Yoruban and African-American samples show a more rapid increase with distance: at 5 kb, 38% of pairs show strong evidence for historical ecombination in both of these two samples, as compared to only 24% and 21% in the European and Asian samples, respectively. Interestingly, beyond the Skb distance, the curves for all four population samples are essentially parallel, indicating a similar incremental chance of encountering historical recombination with larger marker separations (FIG. 9A).

Whether the block-like structure of LD was also evident in the other population samples was also investigated. Applying the block definition described above, strong evidence for blocks in all four population samples was found. At the highest marker density (one highly polymorphic marker every 1 kb), a similar and high proportion of the sequence examined was found in blocks in the European and Asian samples (90% and 88%, respectively), with a lower fraction of sequence in blocks in the Yoruban (75%) and African-American (76%) samples (Table 7). The underlying distribution of block sizes using the two methods described above was determined, and close agreement between the two estimates was obtained. These analyses indicate that the average block size in the two non-African populations is ≈26 kb, while the mean size in the two populations with recent African ancestry is ≈16 kb. In the non-African samples, it is estimated that 88% of the genome is in blocks of 10 kb or larger; in the two samples with recent African ancestry, 76% of the genome sequence is estimated to be in blocks of 10 kb or larger.

Whether the locations and boundaries of blocks were similar across populations was also investigated. The block assignments for pairs of adjacent SNPs in pairs of population samples were studied. An SNP pair was designated as concordant if both members of the pair were contained within a single block in both population samples, or if both members showed strong evidence of historical recombination in the two samples. Similarly, a SNP pair was termed discordant if it was contained within a single block in one population, but showed recombination in the other sample (To maximize power, these comparisons were made for all SNP pairs that were within five to twenty kilobases of one another. At shorter distances, nearly all SNP pairs are in a single block, and at greater distances, many SNP pairs are in different blocks, limiting power.) In the European and Yoruban samples (n=528 pairs), the great majority of SNP pairs (79%) were concordant: 34% were in a single block in both populations, and 45% showed evidence of historical recombination in both populations. Of the pairs that were discordant, furthermore, 81% (83/102) shared a single block in the European sample, but spanned a site of historical recombination in the Yoruban sample. Very similar results were obtained when the Asian sample was compared to the Yoruban sample (n=469 pairs): 74% of pairs were concordant, and of the 26% that were discordant, nearly all (109/124=88%) were contained within a single block in the Asian sample but not in the Yoruban sample.

The rates of concordance were substantially higher for the comparison of the two samples with recent African ancestry: block assignments were concordant 92% of the time when the African-American and the Yoruban samples were compared (n=661 pairs). Similarly, the rates of concordance were higher for the comparison of European and Asian samples (89%, n=537 pairs) than when either of the two is compared with the Yoruban or African-American samples (see above).

In summary, sites of historical recombination (and thus the boundaries between blocks) are highly correlated across all the populations examined.. Even higher rates of similarity are observed when the two samples with recent African ancestry, or the two non-African samples, are compared to one another. These data indicate that the non-African samples tend to manifest a subset of the historical recombination events contained in the samples of recent African ancestry, and generalize patterns proposed in a number of previous reports.

The similarity in recombination sites among populations can be explained by the fact that the history of these populations is largely shared. Moreover, it is likely that the haplotypes that show unambiguous evidence for recombination in the African samples but not in the non-African samples were lost during one or more bottlenecks in the history of the non-African populations. Just as common SNPs are more likely to be pan-ethnic than are rare SNPs.

### EXAMPLE 8

Comparison of Haplotype Diversity Across Populations

The manner in which haplotype diversity (within blocks) varies across populations was also studied. For each block in each population sample, we examined the number of common (≧5%) haplotypes and the proportion of all chromosomes attributable to these haplotypes. Limited haplotype diversity was found to be general across populations, with the number of common haplotypes approaching a plateau when as few as 6-8 common markers have been typed. Examining blocks containing 8-12 markers, there was an average of 3.7 common haplotypes in the Asian samples (capturing 92% of all chromosomes), 4.1 common haplotypes in the European sample (91% of all haplotypes), 4.9 in the Yoruban sample (91% of all haplotypes) and 4.9 in the African-American sample (88% of all haplotypes). That is, the least haplotype diversity is observed in the Asian sample, the next most in the European sample, and the most haplotype diversity in the samples with recent African ancestry.

The specific haplotypes observed when individual blocks are compared across samples from the three continental groups: the European sample, the Asian sample, and the Yoruban (African) sample were compared (Blocks and haplotypes were identified separately in each population sample, and the results compared for blocks that were physically overlappping in all three samples.. On average, there were 5.9 haplotypes that were present at ≧5% frequency in any one of the three samples, of which 46% (2.7) were identified in all three population samples. An additional 29% (1.7) of all haplotypes were observed in two of the three groups (1.0 shared by the European and Yoruban samples, 0.3 shared by the Asian and Yoruban samples, and 0.4 shared by the European and Asian samples,). On average, only 25% (1.5) of all haplotypes were limited to a single population sample, of which 1.0 were seen only in the Yoruban sample, 0.3 in only the Asian sample, and 0.2 in only the European sample. In summary, the vast majority of haplotypes (>75%) are observed in samples from more than one continental group, with the majority of those that are unique to one population being found only in the Yoruban sample.

### EXAMPLE 9

Analysis of Haplotype Blocks Across the Pooled Population Sample

The similarities in the structure and diversity of haplotypes among the four samples suggested that it should be possible to directly detect haplotype blocks in a pooled analysis of all 400 chromosomes. Indeed, when the genotypes from all four population samples were pooled, blocks were readily identified using the same criterion as above, and with size distributions similar to those observed in the Yoruban and African-American samples. In blocks thereby defined, there were on average 5.1 common haplotypes (minor allele frequency ≧5%). The merged analysis directly demonstrates that both the block structure (sites of historical recombination) and specific alleles observed are often shared across population samples, and can be readily identified in a pooled sample. To define the specific characteristics of haplotypes in each sample, however—such as their extent, diversity and allele frequencies—it is necessary to examine each sample individually.

While this invention has been particularly shown and described with references to preferred embodiments thereof, it will be understood by those skilled in the art that various changes in form and details may be made therein without departing from the scope of the invention encompassed by the appended claims.

What is claimed is:

1. A haplotype map of a region of interest of the human genome comprising one or more discrete haplotype blocks bounded by one or more sites of recombination.

2. The map of claim 1, wherein the boundaries of the discrete haplotype blocks are determined by calculating the normalized linkage disequilibrium, D′, of pairs of polymorphic markers.

3. The map of claim 2, wherein 95% confidence intervals of the D′ of the pairs of polymorphic markers are utilized in determining the boundaries of the discrete haplotype blocks.

4. The map of claim 3, wherein the pairs of polymorphic markers have a minor allele frequency of about 0.20.

5. The map of claim 1, wherein the region of interest comprises chromosome 5q31.

6. A method of producing a haplotype map of a region of interest of the human genome comprising determining the pattern of historical recombination across the region of interest and identifying one or more discrete haplotype blocks bounded by one or more sites of recombination, thereby producing a haplotype map of the region of interest.

7. The method of claim 5, wherein the boundaries of the discrete haplotype blocks are determined by calculating the D′ of pairs of polymorphic markers.

8. The method of claim 7, wherein 95% confidence intervals of the D′ of the pairs of polymorphic markers are utilized in determining the boundaries of the discrete haplotype blocks.

9. The method of claim 8, wherein the pairs of polymorphic markers have a minor allele frequency of about 0.20.

10. A method of selecting a set of single nucleotide polymorphic sites (SNPs) for use in genotyping a genomic region of interest comprising the steps of:
identifying at least one SNP which distinguishes each major haplotype in each discrete haplotype block in a haplotype map of the genomic region of interest of the human genome, wherein the haplotype map comprises one or more discrete haplotype blocks bounded by one or more sites of recombination; and
selecting a sufficient number of the SNPs from each discrete haplotype block for use in a genotyping study;
thereby selecting a set of SNPs for use in genotyping studies of the genomic region of interest.

11. A method of identifying an association between a phenotype and a haplotype comprising the steps of:
identifying at least one SNP which distinguishes each major haplotype in each discrete haplotype block in a haplotype map of the genomic region of interest of the human genome, wherein the haplotype map comprises one or more discrete haplotype blocks bounded by one or more sites of recombination;
selecting a sufficient number of the SNPs from each discrete haplotype block to form a set of SNPs suitable for use in a genotyping study; and
assessing the the set of SNPs for an association between a phenotype and a haplotype

12. The method of claim 11, wherein the set of SNPs consists of the sum of the number of major haplotypes in each discrete haplotype block minus the number of discrete haplotype blocks.

13. A method of identifying an association between a phenotype and a haplotype comprising the steps of:
identifying a set of SNPs which uniquely distinguishes a haplotype by selecting the members of the set from the discrete haplotype blocks of a haplotype map consisting of one or more discrete haplotype blocks bounded by one or more sites of recombination; and
assessing the set of SNPs to identify an association between a phenotype and a haplotype.

14. The method of claim 13, wherein the set of SNPs comprises at least one SNP which distinguishes each major haplotype in each discrete haplotype block.

15. A method of identifying the location of a gene associated with a phenotype comprising the following steps:
identifying a set of SNPs which uniquely distinguishes a haplotype by selecting the members of the set from the discrete haplotype blocks of a haplotype map of a chromosomal region associated with the phenotype consisting of one or more discrete haplotype blocks spanned by one or more sites of recombination; and
assessing the set of SNPs to identify an association between a phenotype and a haplotype, wherein identification of the association between the haplotype and the phenotype is indicative of the location of the gene.

16. A method of localizing the position of a disease-susceptibility locus of a disease comprising the steps of:
identifying a set of SNPs which uniquely distinguishes a haplotype by selecting the members of the set from a haplotype map of a chromosomal region associated with the phenotype consisting of one or more discrete haplotype blocks spanned by one or more sites of recombination; and
assessing the set of SNPs to identify an association between the haplotype and the phenotype of the disease, wherein identification of the association between the haplotype and the phenotype is indicative of the position of the disease-susceptibility locus of the disease.

17. A method of diagnosis for susceptibility to a disease comprising the steps of:
identifying a set of SNPs which uniquely distinguishes a haplotype in a chromosomal region associated with the disease by selecting the members of the set from the discrete haplotype blocks of a haplotype map of the chromosomal region consisting of one or more discrete haplotype blocks bounded by one or more sites of recombination; and
assessing the set of SNPs to identify an association between the haplotype and the phenotype of the disease, wherein identification of the association between the haplotype and the phenotype is indicative of susceptibility to the disease.
